(19) United States
(12) Patent Application Publication
(10) Pub. No.: US 2022/0323443 A1
(43) Pub. Date: Oct. 13, 2022

(54) COMBINATION THERAPY FOR CANCER TREATMENT

(71) Applicant: TAKEDA PHARMACEUTICAL COMPANY LIMITED, Osaka (JP)

(72) Inventors: Akihiro Ohashi, Kanagawa (JP); Kenichi Iwai, Kanagawa (JP); Tadahiro Nambu, Kanagawa (JP); Jie Yu, Cambridge, MA (US); Kurt Eng, Cambridge, MA (US); Michael Joseph Kuranda, Cambridge, MA (US); Kazuho Nishimura, Kanagawa (JP); Cong Li, Cambridge, MA (US)

(21) Appl. No.: 17/627,857

(22) PCT Filed: Jul. 17, 2020
(86) PCT No.: PCT/JP2020/028773

§ 371 Date: Jan. 18, 2022

Related U.S. Application Data
(60) Provisional application No. 62/876,167, filed on Jul. 19, 2019.

(51) Int. Cl. A61K 31/519; A61K 31/555; A61K 45/06; A61P 35/00

(52) U.S. Cl. CPC A61K 31/519; A61K 31/555; A61K 45/06; A61P 35/00

(57) ABSTRACT

The present disclosure relates to the treatment of cancer using a combination therapy comprising Compound 1 and/or tautomers thereof or a pharmaceutically acceptable salt or hydrate thereof, and a second therapy.

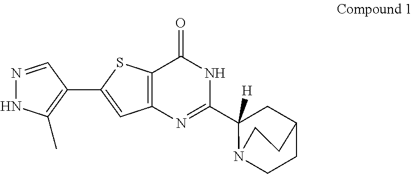 [SMILES: Cc1[nH]ncc1-c1cc2nc([C@@H]3CC4CCN3CC4)[nH]c(=O)c2s1]

### FIELD

The present disclosure relates to the treatment of cancer using a combination therapy comprising (i) Compound 1

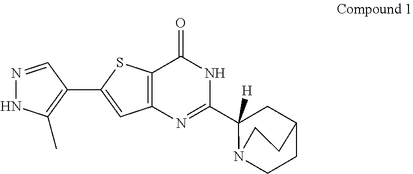 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and (ii) one or more second therapeutic agents and/or a second therapy.

### BACKGROUND

CDC7 is a serine/threonine kinase, which contributes to initiation of DNA replication by phosphorylating MCM2. Kinase activity of CDC7 is controlled by its binding protein Dbf4 in a cell-cycle dependent manner. Recent studies revealed that CDC7 is also involved in DNA damage response (DDR) as well as DNA replication, suggesting that CDC7 plays important roles in both cell proliferation during the S phase and genomic stability in DDR. Furthermore, elevated CDC7 expression has been reported in various cancers and correlates with poor prognosis, such as in diffuse large B cell lymphoma, oral squamous carcinoma, breast tumor, colon tumor, ovarian tumor and lung tumor.

Given that CDC7 is responsible for two key functions of DNA replication and DDR, CDC7 appears to be a critical gene for proliferation and survival of cancer cells and inhibition of CDC7 is expected to induce anti-proliferation and apoptosis in broad range of cancers, not limited to specific organ types of cancers. There is a need for new cancer therapies, such as combination therapies comprising CDC7 inhibitors.

### SUMMARY

The present disclosure provides a method of treating cancer in a patient in need thereof comprising administering a therapeutically effective amount of (i) Compound 1

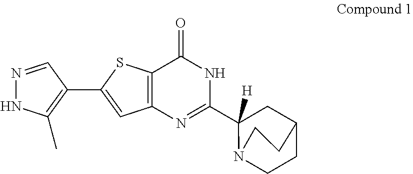 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and (ii) one or more second therapeutic agents and/or a second therapy.

In some embodiments, the second therapeutic agent is selected from a DNA damaging agent, a tubulin binder, a cell signaling modulator, a HSP90 inhibitor, a HDAC inhibitor, a checkpoint inhibitor, an antimetabolite, etoposide, entinostat, obatoclax, and tunicamycin.

In some embodiments, the second therapy is one or more irradiation treatments.

In some embodiments, the present disclosure provides a method of treating cancer in a patient in need thereof comprising administering a therapeutically effective amount of (i) Compound 1, one or more second therapeutic agents and the second therapy (i. e. an irradiation treatment).

The present disclosure also provides pharmaceutical compositions comprising Compound 1 and/or tautomers thereof or a pharmaceutically acceptable salt or hydrate thereof and a second therapeutic agent and uses thereof for treating cancer.

Another aspect of the present disclosure provides a method of determining whether to treat a patient with cancer with Compound 1 and/or tautomers thereof or a pharmaceutically acceptable salt or hydrate thereof,

comprising: (i) determining a mutation and/or deletion status from one or more samples from the patient of one or more gene which selected from a group consisting of ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 and ZNF638; and
(ii) determining to treat the patient with a therapeutically effective amount of Compound 1 and/or tautomers thereof or a pharmaceutically acceptable salt or hydrate thereof if the one or more samples have the mutation and/or deletion of the gene.

Another aspect of the present disclosure provides a method of treating cancer comprising: (i) determining a mutation and/or deletion status from one or more samples from the patient of one or more gene which selected from a group consisting of ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 and ZNF638; and

(ii) administering a therapeutically effective amount of Compound 1 and/or tautomers thereof or a pharmaceutically acceptable salt or hydrate thereof to the patient if the one or more samples (i) have the mutation and/or deletion.

### BRIEF DESCRIPTION OF FIGURES

FIG. 1A shows the homologous recombination (HR) conversion repair. FIGS. 1B and 1C show that Compound 1 suppresses HR repair activity.

FIG. 2A shows 53BP1 foci assays. FIG. 2B shows Compound 1 delays repair of irradiation induced double-strand breaks (DSBs).

FIG. 3 shows that Compound 1 combined with irradiation exhibits strong antitumor activity compared to either single treatment alone against COLO205 human colorectal adenocarcinoma xenograft tumors.

FIG. 4A shows that Compound 1 combined with carboplatin exhibits strong antitumor activity compared to either single treatment alone against PHTXS-13O human primary ovarian cancer xenografts.

FIG. 4B shows that Compound 1 combined with docetaxel exhibits strong antitumor activity compared to either single treatment alone against PHTXM-35Es human primary esophagus cancer xenografts.

FIG. 5A shows that Compound 1 combined with docetaxel exhibits strong antitumor activity compared to either single treatment alone against PHTXM-79Es human primary esophagus cancer xenografts.

FIG. 5B shows that Compound 1 combined with 5-FU or CPT-11 exhibited strong antitumor activity compared to either single treatment alone against PHTXM-79Es human primary esophagus cancer xenografts.

FIG. 6A shows that Compound 1 combined with gemcitabine exhibited strong antitumor activity compared to either single treatment alone against PHTX-249 Pa human primary pancreatic xenografts.

FIG. 6B shows that Compound 1 combined with palbociclib exhibited strong antitumor activity compared to either single treatment alone against PHTXS-13O human primary ovarian cancer xenografts.

FIG. 7 shows in vivo antitumor activity of Compound 1, anti-mPD-1 antibody, anti-mPD-L1, and anti-mCTLA-4 as single agents or combined in female BALB/c mice bearing J558 mouse plasmacytoma tumors.

FIG. 8 shows in vivo antitumor activity of Compound 1, anti-mPD-1 antibody and NKTR-214 as single agents or combined in female BALB/c mice bearing CT26 Mouse syngeneic colon tumor model.

FIG. 9 shows growth inhibition curve of Compound 1 in RNASEH2A KO TK-6 cells and its counter partner parental TK-6 cells.

### DETAILED DESCRIPTION

Unless defined otherwise, all technical and scientific terms used herein have the same meaning as commonly understood by one of ordinary skill in the art to which this disclosure belongs. Accordingly, the following terms are intended to have the following meanings:

As used in the specification and claims, the singular form “a”, “an” and “the” includes plural references unless the context clearly dictates otherwise.

As used herein, “administration” of a disclosed compound encompasses the delivery to a subject of a compound as described herein, or a prodrug or other pharmaceutically acceptable derivative thereof, using any suitable formulation or route of administration, e.g., as described herein. As used herein, “administration” of irradiation treatment encompasses the delivery of radiation to a subject, i.e., as commonly understood in the field of radiation oncology.

As used herein, “effective amount” or “therapeutically effective amount” refers to the amount of a compound or pharmaceutical composition described herein that is sufficient to effect the intended application including, but not limited to, disease treatment, as illustrated below. In some embodiments, the amount is that effective for detectable killing or inhibition of the growth or spread of cancer cells; the size or number of tumors; or other measure of the level, stage, progression or severity of the cancer. The therapeutically effective amount can vary depending upon the intended application (in vitro or in vivo), or the subject and disease condition being treated, e.g., the weight and age of the subject, the severity of the disease condition, the manner of administration and the like, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, “treatment” and “treating”, are used interchangeably herein, and refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, “subject” or “patient” to which administration is contemplated includes, but is not limited to, humans (i.e., a male or female of any age group) or other primates.

The term “comprises or comprising” refers to “includes, but is not limited to”.

The present disclosure provides methods for treating cancer in a patient in need of treatment. The methods comprise administering to a patient in need thereof a therapeutically effective amount of (i) Compound 1

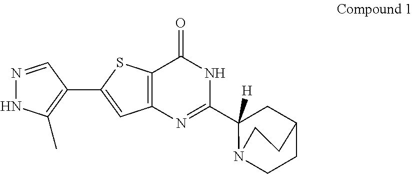 [SMILES: Cc1[nH]ncc1-c1cc2nc([C@@H]3CC4CCN3CC4)[nH]c(=O)c2s1]

and/or tautomers thereof or a pharmaceutically acceptable salt or hydrate thereof, and (ii) one or more second therapeutic agents and/or a second therapy.

The present disclosure further provides a therapeutic combination comprising a therapeutically effective amount of Compound 1 and/or tautomers thereof or a pharmaceutically acceptable salt or hydrate thereof and one or more second therapeutic agents.

The present disclosure further provides a pharmaceutical composition comprising a therapeutically effective amount of Compound 1 and/or tautomers thereof or a pharmaceutically acceptable salt or hydrate thereof and a second therapy.

The present disclosure further provides a pharmaceutical combination comprising a composition comprising Compound 1 and/or tautomers thereof or a pharmaceutically acceptable salt or hydrate thereof and a composition comprising a second therapeutic agent and one or more irradiation treatments.

The present disclosure further provides a kit comprising an article for sale containing a combination comprising Compound 1 and/or tautomers thereof or a pharmaceutically acceptable salt or hydrate thereof and a second therapeutic agent, each separately packaged with instructions for use to treat cancer.

The combination therapies of the present disclosure include Compound 1 and/or tautomers thereof or a pharmaceutically acceptable salt or hydrate thereof. Compound 1 has the following structure:

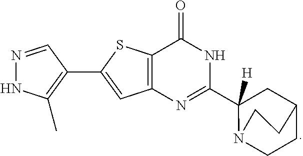 [SMILES: Cc1[nH]ncc1-c1cc2nc([C@@H]3CC4CCN3CC4)[nH]c(=O)c2s1]

The chemical name for Compound 1 is 2-[(2S)-1-azabicyclo[2.2.2]oct-2-yl]-6-(3-methyl-1H-pyrazol-4-yl)thieno[3,2-d]pyrimidin-4(3H)-one. Compound 1 is a CDC7 kinase inhibitor.

CDC7 inhibitors other than Compound 1 are also expected to show good antitumor efficacy in the combination therapies described herein. Thus, in alternative embodiments, the present disclosure further provides a combination therapy comprising a CDC7 kinase inhibitor other than Compound 1. In some embodiments, the CDC7 kinase inhibitor may be selected from LY3143921, KC-459, MSK-777 or RXDX-103. Accordingly, the present disclosure also provides a method for treating cancer in a patient in need thereof, comprising administering to the patient a therapeutically effective amount of a CDC7 kinase inhibitor and one or more second therapeutic agents and/or a second therapy, as described herein.

Tautomers of Compound 1 or a pharmaceutically acceptable salt or hydrate of Compound 1 are/is also encompassed by the present disclosure. When Compound 1 has a tautomer, each isomer is also encompassed in the present disclosure.

As used herein the phrases “Compound 1 and/or tautomers thereof” and the like are all understood to mean Compound 1 and all of its tautomeric forms. As a non-limiting example, tautomerization may occur in the pyrazole and pyrimidine groups of Compound 1. Specific examples of tautomerization that may occur in Compound 1 include:

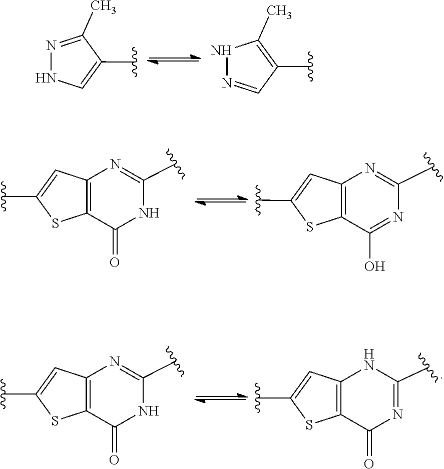

Compound 1 and/or tautomers thereof can be used in the form of a pharmaceutically acceptable salt. Examples of the pharmaceutically acceptable salt include salts with inorganic bases, salts with organic bases, salts with inorganic acids, salts with organic acids, and salts with basic or acidic amino acids.

Compound 1 and/or tautomers thereof may be a hydrate (e.g., hemihydrate), a non-hydrate, a solvate or a non-solvate, all of which are encompassed in the present disclosure. In some embodiments, Compound 1 and/or tautomers thereof is a hemihydrate.

Compound 1 and/or tautomers thereof or a pharmaceutically acceptable salt or hydrate thereof or a crystal form thereof can be obtained according to the production methods described in PCT Publication No. WO 2011/102399, U.S. Pat. Nos. 8,722,660, 8,921,354, 8,933,069, and U.S. Patent Publication No. US 2015/158882, which are incorporated herein by reference in their entirety and for all purposes, or a method analogous thereto.

Compound 1 and/or tautomers thereof or a pharmaceutically acceptable salt or hydrate thereof may be in the form of a crystal (e.g., crystalline form A, crystalline form I, etc.), and the crystal form of the crystal may be single or plural, both of which are encompassed in Compound 1. The crystal may be of a form, and can be produced by a method, described in PCT publication no. WO 2017/172565, published Oct. 5, 2017, which is incorporated herein by reference in its entirety for all purposes. In some embodiments, the Compound 1 and/or tautomers thereof or a pharmaceutically acceptable salt or hydrate thereof may be in the form of Crystalline Form I as described in WO 2017/172565. In some embodiments, the Compound 1 and/or tautomers thereof or a pharmaceutically acceptable salt or hydrate thereof is a crystalline form of Compound 1 hemihydrate (i.e., 2-[(2S)-1-azabicyclo[2.2.2]oct-2-yl]-6-(3-methyl-1H-pyrazol-4-yl)thieno[3,2-d]pyrimidin-4(3H)-one hemihydrate). For example, the Compound 1 and/or tautomers thereof or a pharmaceutically acceptable salt or hydrate thereof may be Crystalline Form I of Compound 1 hemihydrate.

The combination therapy of the present disclosure comprises administration of a second therapeutic agent and/or a second therapy. In some embodiments, the second therapy is irradiation treatment. In some embodiments, the second therapeutic agent is selected from: a DNA damaging agent, a tubulin binder, a cell signaling modulator, a HSP90 inhibitor, a HDAC inhibitor, a checkpoint inhibitor, an antimetabolite, etoposide, entinostat, obatoclax, and tunicamycin.

In some embodiments, the combination therapy comprises a third agent. In some embodiments, the third agent is a therapeutic agent selected from among the second therapeutic agents described herein.

In some embodiments, the second therapeutic agent is an agent having a synergistic effect when used in a combination therapy with Compound 1 and/or tautomers thereof or a pharmaceutically acceptable salt or hydrate thereof. For example, in some embodiments, the second therapeutic agent is a compound or class of compounds reported herein as producing a synergistic effect when used in a combination therapy with Compound 1 and/or tautomers thereof or a pharmaceutically acceptable salt or hydrate thereof.

In some embodiments, the second therapeutic agent is a DNA damaging agent. In some embodiments, the DNA damaging agent is selected from the group consisting of mitomycin C, teniposide, topotecan hydrochloride, carboplatin, decitabine, melphalan, mitoxantrone hydrochloride, irinotecan, cisplatin, oxaliplatin, bleomycin, busulfan, cytarabine, daunorubicin, thiotepa, doxorubicin hydrochloride, gemcitabine, 8-methoxypsoralen, aphidicolin glycinate, brefeldin A, carmustine, chlorambucil, dacarbazine, dactinomycin, mercaptopurine, O6-bezylguanine, SN-38, temozolomide and 5-FU (fluorouracil).

In some embodiments, the DNA damaging agent is selected from the group consisting of myitomycin C, teniposide, topotecan, carboplatin, decitabine, melphalan, mitoxantrone HCl, irinotecan, cisplatin, oxalitplatin, and bleomycin.

In some embodiments, the DNA damaging agent is selected from the group consisting of myitomycin C, teniposide, topotecan, carboplatin, decitabine, and melphalan.

In some embodiments, the DNA damaging agent is selected from the group consisting of topotecan, irinotecan, carboplatin, cisplatin, oxaliplatin, and gemcitabine.

In some embodiments, the DNA damaging agent is selected from the group consisting of carboplatin, 5-FU, irinotecan and gemcitabine.

In some embodiments, the DNA damaging agent is selected from the group consisting of 5-FU, irinotecan and gemcitabine.

In some embodiments, the DNA damaging agent is a topoisomerase inhibitor or a platinum compound.

In some embodiments, the second therapeutic agent is a tubulin binder. In some embodiments, the tubulin binder is selected from docetaxel, paclitaxel, vincristine sulfate and colsemid. In some embodiments, the tubulin binder is docetaxel.

In some embodiments, the second therapeutic agent is a cell signaling modulator. In some embodiments, the cell signaling modulator is selected from alvocidib, BEZ-235, BKM-120, flavopiridol, GDC-0941, PKC412, PLX4032, afatinib, osimertinib, poziotinib, lapatinib, trametinib, cobinetinib, binimrtinib, cobinetinib, binimrtinib, selumetinib, palbociclib, ribociclib, roscovitine, milciclib, dinaciclib, flavopiridol, PHA-793887, AZD5438, BS-181, PF-06873600, KU-55933, KU-60019, VE-821, VE-822, AZD6738, wortmannin, AZD1390, LY2090314, CHI-99021, pictilicib, idelalisib, buparlisib, PI-103, KU-57788, alpelisib, voxtalisib, omipalisib, PF-04691502, AZD6482, GSK1059615, duvelisib, gedatolisib, copanlisib, taselisib, AMG319, seletalisib, pilaralisib, voxtalisib, serabelisib and nemiralisib.

In some embodiments, the cell signaling modulator is selected from GDC-0941, BKM-120, Alvocidib, BEZ-235, Flavopiridol, PKC412, PLX4032 and palbociclib.

In some embodiments, the cell signaling modulator is GDC-0941.

In some embodiments, the second therapeutic agent is HSP90 inhibitor. In some embodiments, the HSP90 inhibitor is selected from 17-AAG, 17-DMAG and AUY-922.

In some embodiments, the second therapeutic agent is HDAC inhibitor. In some embodiments, the HDAC inhibitor is selected from entinostat and panobinostat. In some embodiments, the HDAC inhibitor is entinostat.

In some embodiments, the second therapeutic agent is a checkpoint inhibitor. In some embodiments, the checkpoint inhibitor is selected from an anti-PD-1 antibody, NKTR-214, an anti-CTLA-4 antibody, and an anti-PD-L1 antibody. In some embodiments, NKTR-214 and anti-PD-1 antibody are used as the second therapeutic agent and the third therapeutic agent.

In some embodiments, anti-PD-1 antibody is selected from Nivolumab, Pembrolizumab, Cemiplimab and Spartalizumab.

In some embodiments, anti-CTLA-4 antibody is selected from Ipilimumab and Tremelizumab.

In some embodiments, anti-PD-L1 antibody is selected from Atezolizumab, Durvalumab and Avelumab.

In some embodiments, the second therapeutic agent is etoposide.

In some embodiments, the second therapeutic agent is entinostat.

In some embodiments, the second therapeutic agent is obatoclax.

In some embodiments, the second therapeutic agent is tunicamycin.

In some embodiments, the second therapeutic agent is AT101.

In some embodiments, the second therapeutic agent is azacitidine.

In some embodiments, the second therapeutic agent is bafilomycin A.

In some embodiments, the second therapeutic agent is thapsigargin.

In some embodiments, the second or third therapeutic agent is one or more substances which inhibit gene function of ALKBH6, APEX1, APEX2, ARFGEF1, ASF1A, ASF1B, ATRX, BAZ1B, C21 orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 or ZNF638.

In some embodiments, substance which inhibits gene function includes (i) inhibitor of the gene expression (e.g., anti-sense RNA, siRNA, shRNA) and (ii) inhibitor of protein which translated from the gene (e.g., small molecular compound, antibody).

In some embodiments, the present disclosure provides a method of predicting the likelihood that a patient will respond therapeutically to a cancer treatment comprising the administration of Compound 1 and/or tautomers thereof or a pharmaceutically acceptable salt or hydrate thereof; which comprises determining a mutation and/or deletion status of a sample from a patient of one or more genes which selected from a group consisting of ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 and ZNF638.

In some embodiments, the genes are selected from a group consisting of RNASEH2A, RNASEH2B and RNASEH2C. In some embodiments, the gene is RNASEH2B.

In one embodiment, the method of the present disclosure comprises (1) determining the mutation and/or deletion status, and (2) predicting an increased likelihood that the patient will respond therapeutically to the cancer treatment based on the status in step (1)—specifically, predicting an increased likelihood that the patient will respond therapeutically to the cancer treatment if the sample(s) tests reveal that the one or more genes are mutated and/or deleted.

In one embodiment, the present disclosure provides a method for treating a patient comprising (1) determining whether the patient has the mutation and/or deletion status by (a) obtaining or having obtained a biological sample from the patient; (b) performing or having performed an assay on the biological samples to reveal if the patient has one or more mutated and/or deleted genes; (2) if the patient has the mutation and/or deletion status, then administering a therapeutically effective amount of Compound 1 and/or tautomers thereof or a pharmaceutically acceptable salt or hydrate thereof to the patient; wherein the mutation and/or deletion gene is selected from ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 and ZNF638. In some embodiments, the method further comprises a second therapeutic agent, for example, a DNA damaging agent

Methods, assays, or tests for determining the mutation and/or deletion status are well known in the art. Examples of such method include, but are not limited to, RFLP (Restriction Fragment Length Polymorphism) method, PCR-SSCP (Single Strand DNA Conformation Polymorphism) method, ASO (Allele Specific Oligonucleotide) hybridization method, sequencing method, ARMS (Amplification Refracting Mutation System) method, denaturing gradient gel electrophoresis method, RNAse A cleavage method, DOL (Dye-labeled Oligonucleotide Ligation) method, TaqMan PCR method, primer extension method, invader method, Scorpion-ARMS method, F-PHFA method, pyrosequence method, BEAMing method, RT-PCR, FISH, IHC, immunodetection method, Western Blot, ELISA, radioimmuno assay, immunoprecipitation, FACS, HPLC, surface plasmon resonance, optical spectroscopy, and mass spectrometry. In particular, next generation sequencing methods, e.g., whole exome sequencing (WES) and RNA sequencing (RNASeq) may be used.

Examples of the biological samples used in the methods, assays, or tests include, but are not limited to, serum, whole fresh blood, peripheral blood mononuclear cells, frozen whole blood, fresh plasma, frozen plasma, urine, saliva, skin, hair follicle, bone marrow, tumor tissue, tumor biopsy, or archived paraffin-embedded tumor tissue. The sample is preferably tumor tissue or tumor biopsy comprising cancer cells.

The status of the gene mutation may be, for example, at the level of genomic DNA, protein and/or mRNA transcript of the gene. Preferably, presence or absence of mutation in the gene is determined at the level of genomic DNA or mRNA transcript.

In some embodiments, the combination therapy of the present disclosure may include one or more irradiation treatments. For example, the combination therapy of the present disclosure may comprise administration of Compound 1 and/or tautomers thereof or a pharmaceutically acceptable salt or hydrate thereof and one or more irradiation treatment. Irradiation treatment for treating cancer is well known in the art. See, e.g., Principles and Practice of Radiation Therapy, Washington and Leaver, 4^{th} Ed., 2015. Example 4 of the Examples section, below, describes the treatment of mice with colorectal xenograft tumors that were irradiated at a dose of 3 Gy daily using an X-ray irradiator. The results demonstrate the efficacy of irradiation treatment in combination with Compound 1 treatment.

In some embodiments, Compound 1 and/or tautomers thereof or a pharmaceutically acceptable salt or hydrate thereof and a second therapeutic agent may be formulated as a pharmaceutical composition with pharmaceutically acceptable carriers or diluents as well as any other known adjuvants and excipients in accordance with conventional techniques such as those disclosed in Remington: The Science and Practice of Pharmacy, 19th Edition, Gennaro, Ed., Mack Publishing Co., Easton, Pa., 1995.

Pharmaceutical compositions used in embodiments of the present disclosure may also include diluents, fillers, salts, buffers, detergents (e. g., a nonionic detergent, such as Tween-80), stabilizers (e. g., sugars or protein-free amino acids), preservatives, tissue fixatives, solubilizers, and/or other materials suitable for inclusion in a pharmaceutical composition.

The compounds used in embodiments of the present disclosure may be administered via any suitable route, such as an oral, nasal, inhalable, topical (including buccal, transdermal and sublingual), rectal, vaginal and/or parenteral route.

In certain embodiments, one or more of the compounds used in the present disclosure are administered orally, for example, with an inert diluent or an assimilable edible carrier. The active ingredient may be enclosed in a hard or soft shell gelatin capsule, or compressed into tablets. Pharmaceutical compositions which are suitable for oral administration include ingestible tablets, buccal tablets, troches, capsules, elixirs, suspensions, syrups, wafers, and the like containing such carriers as are known in the art to be appropriate.

In certain embodiments, one or more of the compounds used in the present disclosure are administered parenterally. The phrases “parenteral administration” and “administered parenterally” as used herein mean modes of administration other than enteral and topical administration, usually by injection, and include epidermal, intravenous, intramuscular, intraarterial, intrathecal, intracapsular, intraorbital, intracardiac, intradermal, intraperitoneal, intratendinous, transtracheal, subcutaneous, subcuticular, intraarticular, subcapsular, subarachnoid, intraspinal, intracranial, intrathoracic, epidural and infrasternal injection and infusion.

The methods of this disclosure provide efficacious treatments for patients with cancer. In some embodiments, the cancer treated with the combination therapy of the present disclosure is a cancer mediated by CDC7 (for example, colorectal cancer (e.g., metastatic colorectal cancer), lung cancer (e.g., non-small cell lung cancer (e.g., squamous non-small cell lung cancer (including locally advanced squamous non-small cell lung cancer and metastatic squamous non-small cell lung cancer)), mesothelioma, pancreatic cancer (e.g., metastatic pancreatic cancer), pharyngeal cancer, laryngeal cancer, esophageal cancer (e.g., squamous esophageal cancer), gastric cancer duodenal cancer, small intestinal cancer, breast cancer, ovarian cancer, testis tumor, prostate cancer, liver cancer, thyroid cancer, kidney cancer, uterine cancer, brain tumor, retinoblastoma, skin cancer, bone tumor, urinary bladder cancer, hematologic cancer (e.g., multiple myeloma, leukemia, malignant lymphoma, Hodgkin's disease, chronic bone marrow proliferative disease).

In some embodiments, the cancer treated with the combination therapy of this disclosure is selected from the group consisting of lung cancer (e.g., non-small cell lung cancer (e.g., squamous non-small cell lung cancer including locally advanced squamous non-small cell lung cancer and metastatic squamous non-small cell lung cancer)), colorectal cancer (e.g., metastatic colorectal cancer), ovarian cancer, pancreatic cancer (e.g., metastatic pancreatic cancer), esophagus cancer, prostate cancer, breast cancer, plasmacytoma, hepatoma, melanoma, and lymphoma. In some embodiments, the cancer is selected from lung cancer (e.g., non-small cell lung cancer (e.g., squamous non-small cell lung cancer including locally advanced squamous non-small cell lung cancer and metastatic squamous non-small cell lung cancer)), colorectal cancer (e.g., metastatic colorectal cancer), ovarian cancer, and pancreatic cancer (e.g., metastatic pancreatic cancer).

In some embodiments, the cancer treated with the combination therapy of this disclosure is a platinum compound-resistant cancer.

In some embodiments, the cancer treated with the combination therapy of this disclosure is a cancer of a type that can repair homologous recombination in the cancer cell. A cancer that can repair homologous recombination means the cancer is not HRD (homologous recombination deficient). One example of an HRD cancer is BRCA mutant cancer. There are commercially available kits to test cancer for HRD. One method is to measure the level of expression of one or more human genes involved in the repair of double-stranded DNA breaks from a biological sample from the patient, wherein the biological sample is a tumor cell or tissue from the patient, and wherein the one or more human genes comprise two or more of genes selected from the group consisting of the group of RPA, ATRIP, ATR, Mre 11/Rad50/NBS1, ATM, MDC1, BRCA1, 53BP1, CtIP, Rifl, ku70, ku80, artemis, DNA-pk, XRCC4/Ligase IV, Rad 51, Palb2, BRCA2, RAD52, XRCC3/RAD51C, XRCC2/RAD51B/RAD51D, RAD51AP1, BLM, PAR, RAD54L, RAD54B, Fbhl, WRN, MYC, and STAT3. See, e.g., US 2016/0369353 A1, which is incorporated herein by reference.

In some embodiments, the dose strength of Compound 1 and/or tautomers thereof or a pharmaceutically acceptable salt or hydrate thereof ranges from 5 to 200 mg. For example, in some embodiments, a medicament comprises a dose strength of 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, or 200 mg of Compound 1 and/or tautomers thereof or a pharmaceutically acceptable salt or hydrate thereof. In some embodiments, the daily dose of Compound 1 and/or tautomers thereof or a pharmaceutically acceptable salt or hydrate thereof administered to an adult (body weight about 60 kg) ranges from 10 to 200 mg. In other embodiments, the daily dose to an adult of Compound 1 and/or tautomers thereof or a pharmaceutically acceptable salt or hydrate thereof is about 1 to 1000 mg, about 3 to 300 mg, or about 10 to 200 mg, which can be given in a single administration or administered in 2 or 3 portions a day. In some embodiments, the Compound 1 and/or tautomers thereof or a pharmaceutically acceptable salt or hydrate thereof is administered orally.

In some embodiments, the combination therapy comprises topotecan, wherein topotecan is administered intravenously at a dose from about 0.1 to about 10 mg/m² (e.g., about 0.5 to about 2 mg/m²; or about 1.5 mg/m² or about 0.75 mg/m²).

In some embodiments, the combination therapy comprises carboplatin, wherein carboplatin is administered intravenously at a dose from about 50 mg/m² to about 1000 mg/m² (e.g., from about 100 to about 500 mg/m², or about 300 mg/m²).

In some embodiments, the combination therapy comprises gemcitabine, wherein gemcitabine is administered intravenously at a dose from about 100 to about 5000 mg/m² (e.g., from about 500 to about 2000 mg/m², or about 1000 mg/m²).

In some embodiments, the combination therapy comprises irinotecan, wherein irinotecan is administered intravenously at a dose from about 10 mg/m² to about 500 mg/m² (e.g., from about 50 to about 300 mg/m², or about 125 mg/m² or about 180 mg/m²).

In certain embodiments, the Compound 1 and/or tautomers thereof or a pharmaceutically acceptable salt or hydrate thereof is administered daily, once every two days, once every three days, once every four days, once every five days, once every six days, once a week, once every two weeks, or once every four weeks.

In certain embodiments, the Compound 1 and/or tautomers thereof or a pharmaceutically acceptable salt or hydrate thereof and the second therapy may be administered simultaneously or sequentially in any order. In certain embodiments, they may be administered separately or together in one or more pharmaceutical compositions.

In some embodiments, Compound 1 and/or tautomers thereof or a pharmaceutically acceptable salt or hydrate thereof can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second therapeutic agent to patients with cancer.

In some embodiments, the combination therapy comprises a 14 day cycle wherein Compound 1 and/or tautomers thereof or a pharmaceutically acceptable salt or hydrate thereof is administered once daily on days 1-14 and irradiation treatment is performed on days 1, 2, 3, 8, 9 and 10.

In some embodiments, the combination therapy comprises a 28 day cycle wherein Compound 1 and/or tautomers thereof or a pharmaceutically acceptable salt or hydrate thereof is administered once daily on days 1-28 and topotecan is administered on days 1-5 and 15-19.

In some embodiments, the combination therapy comprises a 28 day cycle wherein Compound 1 and/or tautomers thereof or a pharmaceutically acceptable salt or hydrate thereof is administered once daily on days 1-28 and carboplatin is administered on days 1, 5, 9, 13, 17, 21 and 25 (i.e., every fourth day).

In some embodiments, the combination therapy comprises a 14 day cycle wherein Compound 1 and/or tautomers thereof or a pharmaceutically acceptable salt or hydrate thereof is administered once daily on days 1-14 and carboplatin is administered on days 1, 5, 9 and 13 (i.e., every fourth day).

In some embodiments, the combination therapy comprises a 21 day cycle wherein Compound 1 and/or tautomers thereof or a pharmaceutically acceptable salt or hydrate thereof is administered once daily on days 1-21 and gemcitabine is administered on days 1, 4, 8, 11, 15 and 18 (i.e., twice per week).

In some embodiments, the combination therapy comprises a 21 day cycle wherein Compound 1 and/or tautomers thereof or a pharmaceutically acceptable salt or hydrate thereof is administered once daily on days 1-21 and irinotecan is administered on days 1, 5, 9, 13, 17 and 21 (i.e., every fourth day).

In some embodiments, disclosed herein is a method of treating colorectal cancer in a patient in need thereof, comprising administering a therapeutically effective amount of (i) Compound 1 and/or tautomers thereof or a pharmaceutically acceptable salt or hydrate thereof, and (ii) one or more irradiation treatments.

In some embodiments, disclosed herein is a method of treating ovarian cancer in a patient in need thereof, comprising administering a therapeutically effective amount of (i) Compound 1 and/or tautomers thereof or a pharmaceutically acceptable salt or hydrate thereof, and (ii) carboplatin.

In some embodiments, disclosed herein is a method of treating esophagus cancer in a patient in need thereof, comprising administering a therapeutically effective amount of (i) Compound 1 and/or tautomers thereof or a pharmaceutically acceptable salt or hydrate thereof, and (ii) docetaxel.

In some embodiments, disclosed herein is a method of treating esophagus cancer in a patient in need thereof, comprising administering a therapeutically effective amount of (i) Compound 1 and/or tautomers thereof or a pharmaceutically acceptable salt or hydrate thereof, and (ii) 5-FU or CPT-11.

In some embodiments, disclosed herein is a method of treating pancreatic cancer in a patient in need thereof, comprising administering a therapeutically effective amount of (i) Compound 1 and/or tautomers thereof or a pharmaceutically acceptable salt or hydrate thereof, and (ii) gemcitabine.

In some embodiments, disclosed herein is a method of treating plasmacytoma in a patient in need thereof, comprising administering a therapeutically effective amount of (i) Compound 1 and/or tautomers thereof or a pharmaceutically acceptable salt or hydrate thereof, and (ii) an anti-mPD-1 antibody, an anti-mPD-L1 antibody, or an anti-mCTLA-4 antibody.

In some embodiments, disclosed herein is a method of treating colon cancer in a patient in need thereof, comprising administering a therapeutically effective amount of (i) Compound 1 and/or tautomers thereof or a pharmaceutically acceptable salt or hydrate thereof, and (ii) an anti-mPD-1 antibody and/or NKTR-214.

### EXAMPLES

### Example 1: In Vitro Study

To identify agents which enhance anti-proliferative activity of Compound 1, in vitro combination studies of various agents with Compound 1 were carried out in COLO205, A549, SW620, SW48, H460, and HCT116 cancer cells using a fully automated system for assay execution and data analysis. Using adenosine 5′-triphosphate (ATP) as a measure of cell viability, combinations with Compound 1 were classified as synergistic, additive, sub-additive, or antagonistic based on their anti-proliferative effects. Combination performance was ranked based on the most frequent occurrence of synergy across the 6 cell lines tested.

Stock solutions of Compound 1 were prepared in dimethyl sulfoxide (DMSO). Serially diluted stock solutions (0.003 to 200 μM) were stored at approximately 4 deg C.

The cell lines used in Example 1 are listed in Table 1.

TABLE 1
Tumor Cell Lines Used for In Vitro Combination Studies

| | | | Starting | |
|---|---|---|---|---|
| | | | Density | |
| Tumor | | | (×10³ | |
| Cell | Tissue | | cells/ | |
| Line | Origin^{a} | Vendor | well)^{b} | Growth Medium^{c} |
| A549 | Lung | ATCC | 1000 | F12K (Invitrogen, Carlsbad, |
| | | | | CA, USA), 10% FBS, 2 mM |
| | | | | L-Glu, 1× Pen/Strep |
| COLO205 | Colon | ATCC | 1200 | RPMI-1640 (ATCC, |
| | | | | Manassas, VA, USA), 10% |
| | | | | FBS, 2 mM L-Glu, 1× |
| | | | | Pen/Strep |
| H460 | Lung | ATCC | 800 | RPMI (Invitrogen, Carlsbad, |
| | | | | CA, USA), 10% FBS, 2 mM |
| | | | | L-Glu, 1× Pen/Strep |
| HCT116 | Colon | ATCC | 400 | DMEM (Invitrogen, |
| | | | | Carlsbad, CA, USA), 10% |
| | | | | FBS, 2 mM L-Glu, 1× |
| | | | | Pen/Strep |
| SW48 | Colon | ATCC | 800 | McCoy's 5A (Invitrogen, |
| | | | | Carlsbad, CA, USA), 10% |
| | | | | FBS, 2 mM L-Glu, 1× |
| | | | | Pen/Strep |
| SW620 | Colon | ATCC | 800 | Leibovitz's L15 (Invitrogen, |
| | | | | Carlsbad, CA, USA), 10% |
| | | | | FBS, 2 mM L-Glu, 1× |
| | | | | Pen/Strep |

ATCC = American Type Culture Collection;
DMEM = Dulbelcco's Modified Eagle Medium;
F12K = Ham's F-12K (Kaighn's) Medium;
Glu = glutamine;
RPMI = Roswell Park Memorial Institute;
Pen/strep = Penicillin/Streptomycin
^{a}Histological origin of tumors from which cell lines were derived.
^{b}Number of cells plated to ensure optimal linear growth over 72 hours.
^{c}Growth media used to culture tumor cells.

Each combination pair was evaluated in an individual 384-well plate which contained variable doses of both compounds as single agents, as well as two, ten-by-ten matrices (in duplicate) that contained mixtures of the two test compounds. In brief, compounds were added to the cell test plates 16 hours after cell plating and then assessed for viability 72 hours later. Continuous cultures of tumor cells were maintained under standard cell culturing conditions (i.e. in a humidified chamber set at 37° C., containing atmosphere 5% carbon dioxide). After cell counting, cells were plated into assay plates in 254 cell culture media. Seventy-two hours after compound addition, ATP levels were measured to assess cell viability. Plating densities were chosen to ensure optimal linear growth over the 72 hour period.

Compound dilution and compound delivery to the assay plates were done on HighRes Robotic System (HighRes Biosolutions, Woburn, Mass., USA) with Echo™ Liquid Handler (Labcyte, Sunnyvale, Calif., USA). First, 384-well low dead volume (LDV) plates containing DMSO (Appendix B) and 10 mM compound stock solutions were used to create the required intermediate compound dilution plates. Next, these were used for compound transfer into cell assay plates. All wells were back-filled to give a constant percentage of DMSO.

The final DMSO concentration was held constant in all wells across the plate and was maintained at less than 0.5%. Preliminary studies show that at 0.5% DMSO, there is no discernable difference in growth rate compared to cells grown without any DMSO. Dose concentrations of each targeted agent ranged from inactive to maximally effective (defined as causing maximum growth inhibition). These cell viability datasets were used to calculate single agent concentration producing 50% efficacy (EC₅₀) values and classify the inhibitor combination response. Cells treated with vehicle (DMSO) in two rows, or single compound serial dilutions in one plate columns/row served as the untreated control and single compound controls, respectively.

### Cell Titer-Glo® Cell Proliferation Assay

Compound activity in A549, COLO205, H460, HCT116, SW48, SW620 cancer cell lines was assessed using Cell Titer-Glo® (Promega [Madison, Wis., USA]). After 72 hours incubation, the plates were treated as per package insert protocol for the Promega Luminescence ATP Detection Systems. Briefly, 25 μL of cell lysis/substrate solution (provided in kit form) was added to each well and the plate was incubated at room temperature for 10 minutes. Luminescence was measured using a PHERAstar multi-label counter (BMG Labtech [Ortenberg, Germany]) or LEADseeker (GE Healthcare Life Sciences [Piscataway, N.J., USA]).

### Data Analysis ATPlite™ Cell Proliferation Assay

Numerical luminescence values were analyzed to generate EC₅₀ curves and evaluate synergy. Raw Data reader files were uploaded along with automation workfiles that defined the plate and well contents. Percent activities were calculated for each well versus plate controls.

### Statistics

Each plate representing a single drug combination was analyzed separately. First, the viability measurements were normalized by scaling the data so that the median of the negative controls was 0 and the median of the positive controls was 100. Some of the wells on the plate contained only one drug, and this data was used to compute the single drug EC₅₀'s by fitting this data to the Hill equation.

For the combination analysis, a response surface model was used to describe the relationship between the normalized viability and the drug concentrations. The data were fit to the model by minimizing the residual sum of squares. Based on the fitted response surface, plots of constant viability, called isobolograms, were produced.

The Combination Index and Nonlinear Blending were used as measures of drug synergy. To calculate Combination Index the 50% isobologram (which is the dose contour that has 50% viability) was used. The standard error was used for both of these measures using the Cramer-Rao lower bound. A standard procedure was created to produce a call in order to characterize the viability effects for each combination (synergy, additivity, subadditivity, or antagonism). If the Combination Index existed then these measures were used to make the call. If the Combination Index did not exist because one or both or the compounds did not achieve a 50% reduction of viability then a similar procedure based on Nonlinear Blending was used to make the call. Tables 2 and 3 indicate how these calls were made.

TABLE 2
Interpreting the Combination Index

| | Combination | |
|---|---|---|
| P-value | Index | Call |
| >0.05 | Any | Inconclusive |
| <0.05 | 0.7 to 1.3 | Additivity |
| <0.05 | 0 to 0.7 | Synergy |
| <0.05 | 1.3 to 2 | Subadditivity |
| <0.05 | >2 | Antagonism |

TABLE 3
Interpreting Nonlinear Blending

| | Nonlinear | |
|---|---|---|
| P-value | Blending | Call |
| >0.05 | Any | Inconclusive |
| <0.05 | −20 to 20 | Additivity |
| <0.05 | >20 | Synergy |
| <0.05 | <−20 | Antagonism |

Table 4 shows the results of the anti-proliferative activity of the combinations of Compound 1 and DNA damaging agents tested. These studies revealed that, in combination with Compound 1, DNA damaging agents like topoisomerase inhibitors and platinum compounds had the highest occurrence of synergistic anti-proliferative effects.

TABLE 4
Results of Combining Compound 1 with DNA Damaging Agents

| Drug | A549 | COLO205 | H460 | HCT116 | SW48 | SW620 |
|---|---|---|---|---|---|---|
| Mitomycin C | Synergy | Subadditivity | Synergy | Synergy | Synergy | Synergy |
| Teniposide | Synergy | Synergy | Synergy | Synergy | Synergy | Synergy |
| Topotecan | Synergy | Synergy | Synergy | Synergy | Additivity | Synergy |
| hydrochloride | | | | | | |
| Carboplatin | — | — | Synergy | Synergy | Additivity | — |
| Decitabine | — | — | Synergy | Synergy | Additivity | Additivity |
| Melphalan | Synergy | — | Synergy | Synergy | Additivity | Synergy |
| Mitoxantrone | Synergy | Synergy | Synergy | Synergy | Additivity | Synergy |
| hydrochloride | | | | | | |
| SN-38 | Synergy | Additivity | Synergy | Synergy | Additivity | Synergy |
| Cisplatin | — | — | Synergy | Synergy | Additivity | Synergy |
| Oxaliplatin | — | Synergy | Additivity | Additivity | Additivity | Synergy |
| bleomycin | Additivity | Synergy | Synergy | Synergy | Additivity | Additivity |
| Busulfan | — | Synergy | — | — | — | — |
| Aphidicolin | Additivity | Additivity | Additivity | Synergy | — | Additivity |
| glycinate | | | | | | |
| Cytarabine | Additivity | Synergy | Additivity | Additivity | — | Synergy |
| Daunorubicin | Additivity | Additivity | Additivity | Synergy | Additivity | Synergy |
| Thio-tepa | Additivity | — | Additivity | Synergy | Additivity | Synergy |
| Doxorubicin | Synergy | Synergy | Synergy | Subadditivity | — | Additivity |
| hydrochloride | | | | | | |
| Brefeldin A | Additivity | Subadditivity | Additivity | Additivity | — | Additivity |
| Chlorambucil | Additivity | — | Additivity | Additivity | Additivity | — |
| Clofarabine | Subadditivity | Additivity | Subadditivity | Additivity | — | Antagonism |
| Dacarbazine | — | — | — | Additivity | — | — |
| Dactinomycin | Additivity | Additivity | Additivity | Additivity | — | Additivity |
| Gemcitabine | Antagonism | Additivity | Subadditivity | Additivity | — | Additivity |
| Mercaptopurine | — | Additivity | Additivity | Additivity | — | Subadditivity |
| O6-Benzylguanine | — | Additivity | — | — | — | — |

Table 4: Combinations are ordered based on occurrence of synergy in multiple cell lines. All experiments labeled indeterminable or inconclusive were repeated at least twice. Indeterminable refers to poor data quality likely inherent to a particular cell line or compound used. Inconclusive refers to the inability to make a call based on statistical criteria. Combination results of “ [symbol] ” were not run in this study.

Table 5 shows the results of the anti-proliferative activity of the combinations of Compound 1 and Tubulin binders etc. tested. These studies revealed that, in combination with Compound 1, these agents show synergistic or additive anti-proliferative effects etc. in certain conditions.

TABLE 5
Results of Combination study of Compound 1

| Mechanism | Drug | A549 | COLO205 | H460 | HCT116 | SW48 | SW620 |
|---|---|---|---|---|---|---|---|
| Autophagy | ATG7 | Additivity | Additivity | Additivity | Additivity | Antagonism | Subadditivity |
| inhibitor | | | | | | | |
| cell | GDC-0941 | Additivity | Additivity | Synergy | Synergy | — | Additivity |
| signaling | BKM-120 | Additivity | Additivity | Additivity | Additivity | — | Additivity |
| modulator | Alvocidib | Additivity | Additivity | Additivity | Additivity | — | Subadditivity |
| | BEZ-235 | Additivity | Additivity | Additivity | Additivity | Antagonism | Subadditivity |
| | Flavopiridol | — | — | Additivity | — | — | — |
| | PKC412 | — | Subadditivity | Antagonism | _Additivity | — | Additivity |
| | PLX4032 | Additivity | Additivity | Additivity | Additivity | — | — |
| tubulin | Docetaxel | Subadditivity | Additivity | Additivity | Additivity | — | Synergy |
| binders | Paclitaxel | Subadditivity | Additivity | Additivity | Additivity | Antagonism | Additivity |
| | Vincristine | Subadditivity | Additivity | Additivity | Additivity | — | Subadditivity |
| | sulfate | | | | | | |
| other | Etoposide | Synergy | Synergy | Synergy | Synergy | Synergy | Synergy |
| targeted | Obatoclax | Additivity | Additivity | Synergy | Additivity | Additivity | Synergy |
| agents | Tunicamycin | — | Additivity | Additivity | Synergy | — | Additivity |
| | Entinostat | Synergy | Subadditivity | Synergy | Additivity | Subadditivity | Synergy |
| | AUY-922 | Additivity | Additivity | — | Additivity | — | Additivity |
| | AT101 | Additivity | Additivity | Additivity | Additivity | — | Additivity |
| | 17-AAG | Additivity | Additivity | Additivity | Additivity | — | Additivity |
| | 17-DMAG | Subadditivity | Additivity | Additivity | Additivity | — | Additivity |
| | Azacitidine | Additivity | Additivity | Additivity | Additivity | Additivity | Additivity |
| | Bafilomycin A | Additivity | Subadditivity | Additivity | Additivity | — | Additivity |
| | Panobinostat | Additivity | Subadditivity | Additivity | Additivity | — | Additivity |
| | Thapsigargin | Additivity | Additivity | Additivity | Additivity | — | Additivity |

### Example 2: In Vitro Study for Selected Compounds, Additional Cell Lines

In vitro anti-proliferative effects of selected compounds were tested in additional cell lines, including ovarian cancer (SKOV3) and pancreatic cancer (MIA-PACA-2) cell lines. The studies revealed that topoisomerase inhibitors and DNA cross-linker agents induce additive or synergistic effects to Compound 1 in several cancer cell lines. The results are shown in Table 6.

TABLE 6
In Vitro Combination Study for the Selected Compound

| | | | | | Pancreas |
|---|---|---|---|---|---|
| | | Lung | Lung | Ovary | MIA- |
| Mechanism | Drug | CALU6 | H-1650 | SKOV3 | PACA-2 |
| DNA | Topotecan | — | Additive | Additive | Additive |
| damaging | SN-38 | — | Additive | Additive | Additive |
| agent | (Irinotecan) | | | | |
| | Carboplatin | Additive | Additive | — | — |
| | Cisplatin | Additive | Additive | Additive | Sub- |
| | | | | | additive |
| | Oxaliplatin | Additive | Additive | Additive | — |
| | Gemcitabine | Additive | Antagonism | Additive | Sub- |
| | | | | | additive |
| | Fluorouracil | Synergy | Additive | Additive | Additive |
| Tubulin | Docetaxel | — | — | — | — |
| binder | Paclitaxel | Additive | Antagonism | — | Sub- |
| | | | | | additive |

### Example 3A: Compound 1 Suppresses Homologous Recombination (HR) Repair Activity

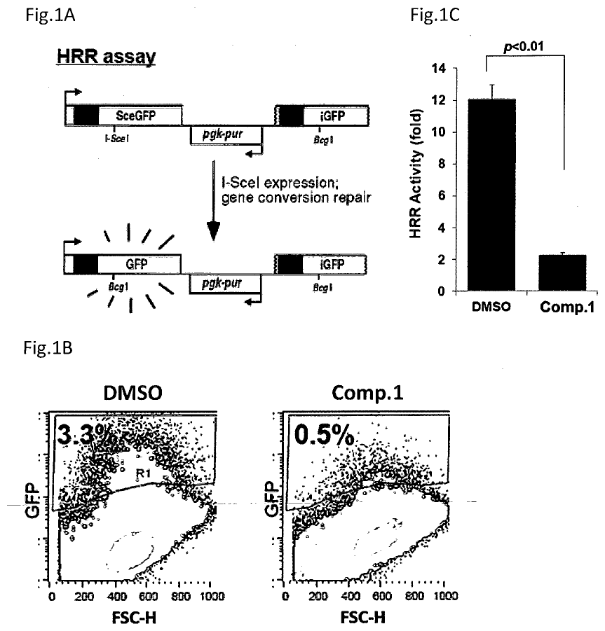

The efficiency of homologous recombination (HR) was assessed using an I-SceI expression plasmid (I-SceI) and an I-SceI repair reporter plasmid (DR-GFP) composed of two differentially mutated GFP genes, one of which contained a unique I-SceI restriction site (FIG. 1A). The assay works through gene conversion repair of a double strand break caused by I-SceI digestion. DR-GFP plasmids repaired by homologous recombination express GFP. Human embryonic kidney 293T cells were transfected with either 5 μg of DR-GFP plus 10 μg of I-SceI in presence (300 nM) or absence of Compound 1. Seventy-two hours after transfection, the cells were fixed with 4% paraformaldehyde for 20 min at room temperature, and the number of GFP-expressing cells was assessed by flow cytometry (FIGS. 1B and 1C).

These results indicate that Compound 1 suppresses HR repair activity.

### Example 3B: Compound 1 Delays Repair of Irradiation (IR)-Induced DNA Double-Strand Breaks (DSBs)

Human cervical adenocarcinoma HeLa cells were treated with or without Compound 1 treatment at 300 nM, followed by irradiation (IR) treatment at 4 Gy using an X-ray irradiator (MBR-1520R-3, Hitachi Power Solutions Co., Ltd., Ibaraki). Eight or forty-eight hours after IR treatment, the cells were fixed with 4% paraformaldehyde for the following immunofluorescent experiments. Foci formation of 53BP1 was used as an index of IR-induced DSBs. After permeabilization, the cells were incubated with anti-53BP1 antibody (2 μg/ml) for 60 min at 37° C., and then incubated with Alexa-594-conjugated secondary antibody for 30 min at 37° C. Images were captured with an Axiovert 200M microscope (Carl Zeiss).

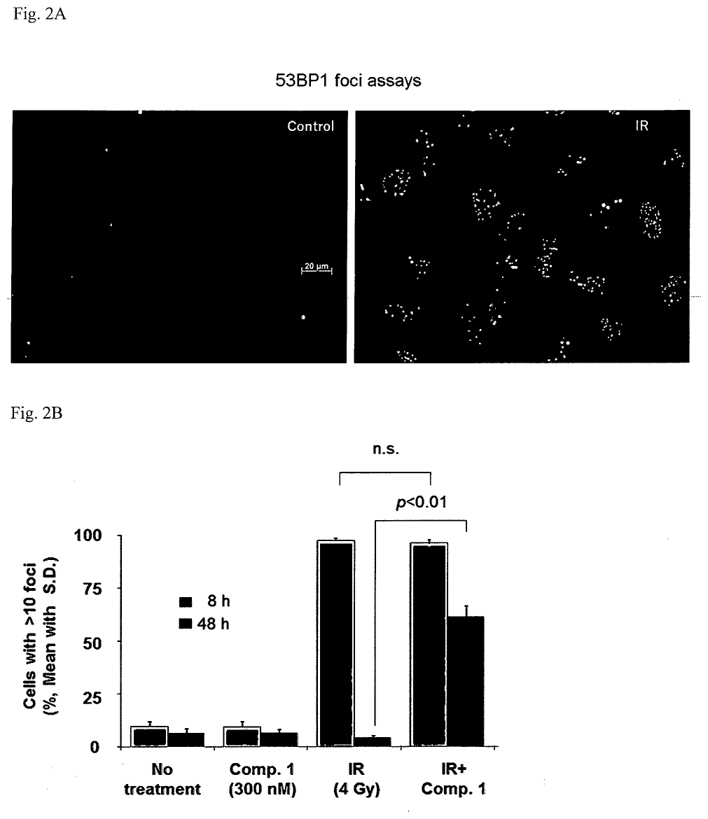

In the cells treated with IR alone, the 53BP1 foci-positive cells (≥10 foci/cell) were drastically increased 8 h after IR treatment, while the foci positive cells were decreased at the equivalent level to the no-treatment cells, indicating that DNA repair was being completed in 48 h after IR treatment (FIG. 2A and FIG. 2B). In the cells co-treated with IR and Compound 1, on the contrary, the 53BP1 foci-positive cells were still observed with high frequency 48 h after IR treatment. These data suggest that Compound 1 delays repair of IR-induced DSBs (FIG. 2B).

Based on the results of Examples 2A and 2B, it was hypothesized that the combination of Compound 1 and a DNA damaging agent could work synergistically for treating cancer.

### Example 4: In Vivo Antitumor Activity of Compound 1 and Irradiation as Single Agents and in Combination in Nude Mice Bearing COLO205 Human Colorectal Adenocarcinoma Xenografts

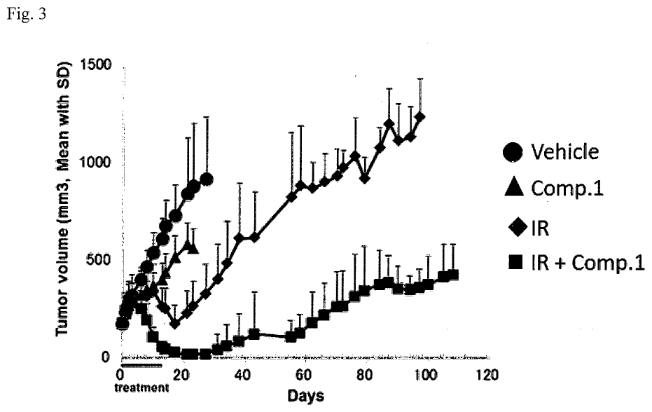

Human colorectal carcinoma cell line, COLO205 xenograft model was established by subcutaneous injection of cell suspension (5×10⁶ cells/100 μl/site, in 1:1 mixture of Hanks' balanced salt and BD Matrigel™ Matrix (BD biosciences)). Mice with tumor size of approximately 200 mm³ were randomly assigned to dose groups on the day before start date of dosing (Day 0). Compound 1 was suspended in 0.5 w/v % methylcellulose and administered orally to mice at a dose of 40 mg/kg once daily on Day 1-14. Mice in irradiation groups were irradiated at a dose of 3 Gy daily on Day 1, 2, 3, 8, 9 and 10 under pentobarbital anesthesia. Tumors on the flanks of the mice were irradiated using an X-ray irradiator (MBR-1520R-3, Hitachi Power Solutions Co., Ltd., Ibaraki) and non-tumor part of mice were shielded by lead plate. Tumor size was measured by caliper and tumor volume was estimated using the equation V=(LW²)/2, where L and W are tumor length and width, respectively and reported in cubic millimeters (FIG. 3). The results of this study demonstrate that Compound 1 combined with irradiation exhibited strong antitumor activity and enhanced antitumor efficacy compared to either single treatment alone against COLO205 human colorectal adenocarcinoma xenograft tumors.

### Example 5: In Vivo Antitumor Activity of Compound 1 in Combination with Other Agents in Cell Derived Xenograft (CDX), Patient Derived Xenograft (PDX) and Syngeneic Mouse Tumor Isograft Model

To investigate in vivo antitumor activity of Compound 1 in combination with other agents, tests using Cell Derived Xenograft (CDX), Patient Derived Xenograft (PDX) and Syngeneic mouse tumor isograft model were conducted. Cells or Patient Derived Tumors were inoculated by one method of the following two methods (Method A and B) as shown Table 7.

Method A; Cells were maintained in either immune deficient nude mice or immune competent mice by subcutaneous inoculation of tumor cells at various concentrations into respected mice.
Method B: Patient Derived Tumor s were maintained in nude mice by subcutaneous inoculation of tumor pieces (approx. 2×2×2 mm) into nude mice. Mice with tumor size of approximately 50 mm³ (e.g., 40-75 mm³) for syngeneic mouse studies or 200 mm³ (e.g., 110-270 mm³) for xenograft studies were randomly assigned to dose groups on the day (Day 0) before start date of dosing.

Compound 1 (crystalline form I) was suspended in 0.5 w/v % methylcellulose and administered orally to mice. Antibodies which administered in the experiment were described in Table 8.

Concomitant drug was administered as shown in Table 9.

Tumor size was measured by caliper and tumor volume was estimated using the equation V=(LW²)/2, where L and W are tumor length and width, respectively and reported in cubic millimeters.

Statistical analyses of combination effect for tumor growth was conducted as follows; All tumor values (tumor volumes or photon flux) had a value of 1 added to them before log₁₀ transformation. These values were compared across treatment groups to assess whether the differences in the trends over time were statistically significant. To compare pairs of treatment groups, the following mixed-effects linear regression model was fit to the data using the maximum likelihood method:

Yᵢⱼₖ−Yᵢ₀ₖ=Yᵢ₀ₖtreatᵢ+dayⱼdayⱼ²+(treat*day)ᵢⱼ+(treat*day²)ᵢⱼeᵢⱼₖ (1)

where Yijk is the log₁₀ tumor value at the j^{th} time point of the k^{th} animal in the i^{th} treatment, Yᵢ₀ₖ is the day 0 (baseline) login tumor value in the k^{th} animal in the i^{th} treatment, dayⱼ was the median-centered time point and (along with day²ⱼ) was treated as a continuous variable, and eᵢⱼₖ is the residual error. A spatial power law covariance matrix was used to account for the repeated measurements on the same animal over time. Interaction terms as well as day²ⱼ terms were removed if they were not statistically significant.

A likelihood ratio test was used to assess whether a given pair of treatment groups exhibited differences which were statistically significant. The −2 log likelihood of the full model was compared to one without any treatment terms (reduced model) and the difference in the values was tested using a Chi-squared test. The degrees of freedom of the test were calculated as the difference between the degrees of freedom of the full model and that of the reduced model.

The predicted differences in the log tumor values (Yᵢⱼₖ−Yᵢ₀ₖ, which can be interpreted as log₁₀ (fold change from day 0)) were taken from the above models to calculate mean AUC values for each treatment group. A dAUC value was then calculated as:

$$d A U C = \frac{mean ( A U {C}_{c t l} ) - mean ( A U {C}_{t r t} )}{mean ( A U {C}_{c t l} )} * 100 \qquad ( 2 )$$

This assumed AUC_{ctl} was positive. In instances where AUC_{ctl} was negative, the above formula was multiplied by −1.

For synergy analyses, the observed differences in the log tumor values were used to calculate AUC values for each animal. In instances when an animal in a treatment group was removed from the study, the last observed tumor value was carried forward through all subsequent time points. The AUC for the control, or vehicle, group was calculated using the predicted values from the pairwise models described above. We defined a measure of synergy as follows:

$${Frac}_{{A}_{k}} = \frac{A U {C}_{ctl} - A U {C}_{{A}_{k}}}{A U {C}_{c t l}} \qquad ( 3 )$$

$${Frac}_{{B}_{k}} = \frac{A U {C}_{c t l} - A U {C}_{{B}_{k}}}{A U {C}_{ctl}} \qquad ( 4 )$$

$${Frac}_{{AB}_{k}} = \frac{A U {C}_{ctl} - A U {C}_{A {B}_{k}}}{A U {C}_{c t l}} \qquad ( 5 )$$

$$synergy score = ( mean ( {Frac}_{A} ) + mean ( {Frac}_{B} ) - mean ( {Frac}_{AB} ) ) * 100 \qquad ( 6 )$$

where Aₖ and Bₖ are the k^{th} animal in the individual treatment groups and ABₖ is the k^{th} animal in combination treatment group. AUC_{cd} is the model-predicted AUC for the control group and was treated as a constant with no variability. The standard error of the synergy score was calculated as the square root of the sum of squared standard errors across groups A, B, and AB. The degrees of freedom were estimated using the Welch-Satterthwaite equation. A hypothesis test was performed to determine if the synergy score differed from 0. P values were calculated by dividing the synergy score by its standard error and tested against a t-distribution
(two-tailed) with the above-calculated degrees of freedom.

The effect was classified into four different categories. It was considered synergistic if the synergy score was less than 0 and additive if the synergy score wasn't statistically different from 0. If the synergy score was greater than zero, but the mean AUC for the combination was lower than the lowest mean AUC among the two single agent treatments, then the combination was sub-additive. If the synergy score was greater than zero, and the mean AUC for the combination was greater than the mean AUC for at least one of the single agent treatments, then the combination was antagonistic.

Interval analysis, if requested, involved a specified treatment group and time interval compared with another treatment group and time interval. For a given group, time interval, and animal, the tumor growth rate per day was estimated by

Rate=100*(10^{ΔY/Δt}−1) (7)

where ΔY is the difference in the login tumor volume over the interval of interest, and Δt is the length of the time interval. If one or both of the time points were missing, then the animal was ignored. The mean rates across the animals were then compared using a two-sided unpaired t-test with unequal variances.

Given the exploratory nature of this study, there were no adjustments pre-specified for the multiple comparisons and endpoints examined. All P values <0.05 were called statistically significant in this analysis.

The results of this study were shown in Table 9.

TABLE 7
Inoculated cells in vivo study

| Name of Cell | Inoculation | |
|---|---|---|
| (Cancer type) | method | Provider |
| PHTXM-97Pa | B | Shanghai Outdo Biotech |
| (Pancreatic) | | Co., Ltd |
| PHTX-249Pa | B | National Disease Research |
| (Pancreatic) | | Institute, Philadelphia |
| PHTXM-35Es | B | Shanghai Outdo Biotech |
| (Esophagus) | | Co., Ltd |
| PHTXM-90Es | B | Shanghai Outdo Biotech |
| (Esophagus) | | Co., Ltd |
| PHTXM-79Es | B | Shanghai Outdo Biotech |
| (Esophagus) | | Co., Ltd |
| PHTX-09C | B | The Ohio State University |
| (Colorectal) | | Medical Center |
| | | (Columbus, OH, USA) |
| PHTXS-13O | B | Hokkaido University |
| (Ovarian) | | (Sapporo, Japan) |
| DU-145 (Prostate) | A | American Type Culture |
| | | Collection (ATCC) |
| CT26 (Colorectal) | A | ATCC |
| JC (Breast) | A | ATCC |
| J558 (Plasmacytoma) | A | ATCC |
| H22 (Hepatoma) | A | ATCC |
| B16F10 (Melanoma) | A | ATCC |
| A20 (Lymphoma) | A | ATCC |
| Panc02 (Pancreatic) | A | ATCC |

TABLE 8
Administered antibody

| Antibody | Clone | Manufacturer |
|---|---|---|
| Anti-mPD-1 | RMP1-14 | BioXCell (West Lebanon, NH, USA) |
| Anti-mPD-L1 | 10F.9G2 | BioXCell (West Lebanon, NH, USA) |
| Anti-mCTLA-4 | 9H10 | BioXCell (West Lebanon, NH, USA) |

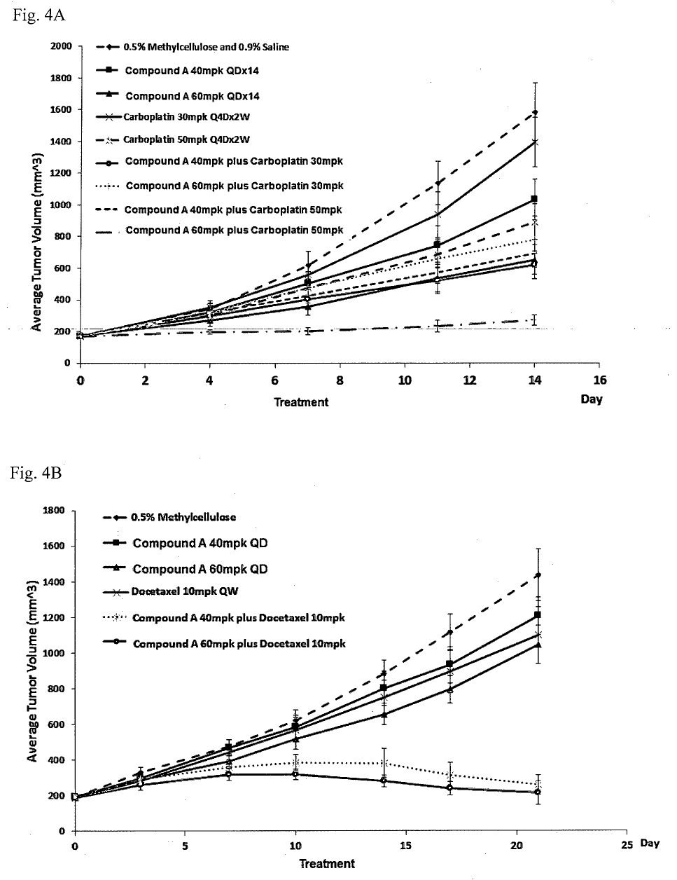

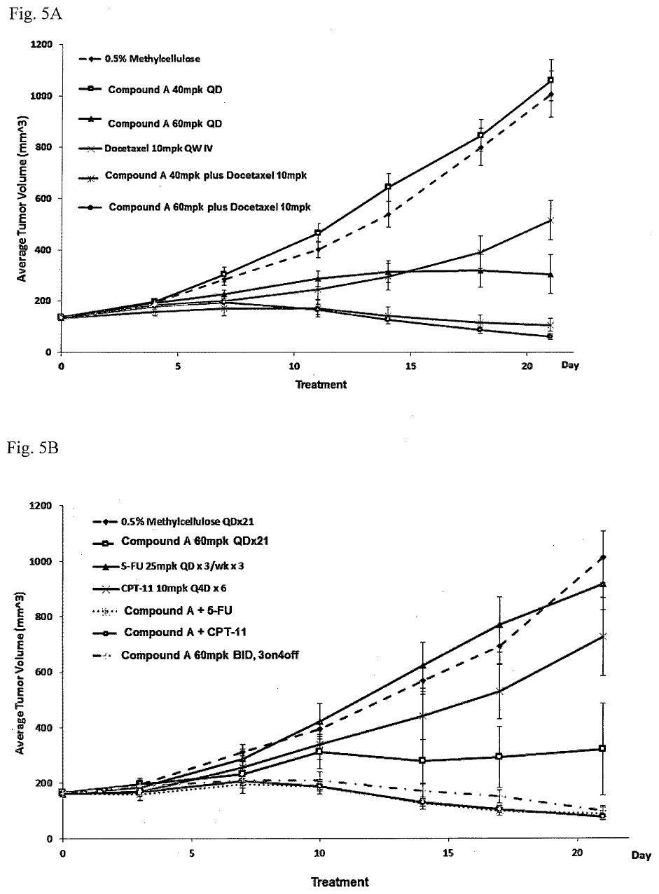

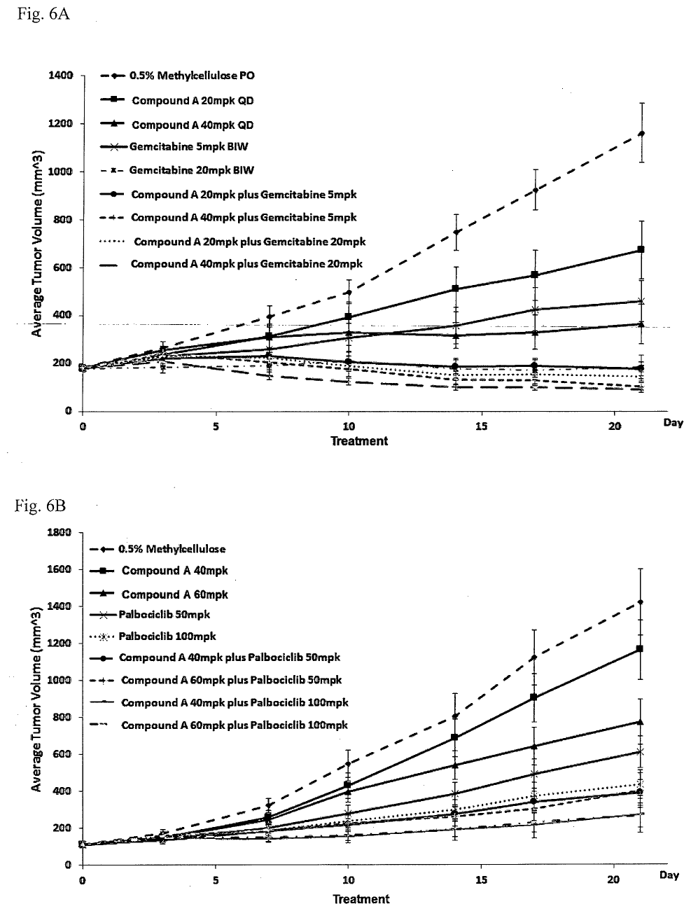

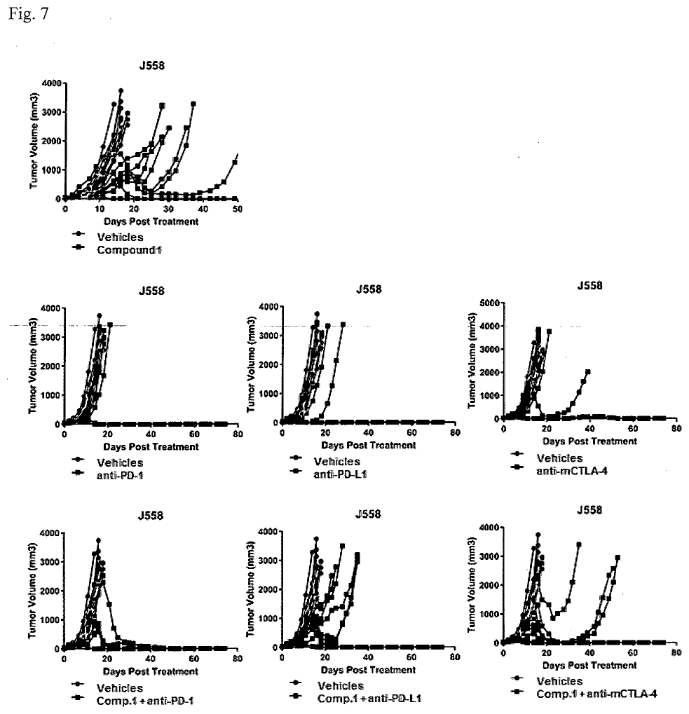

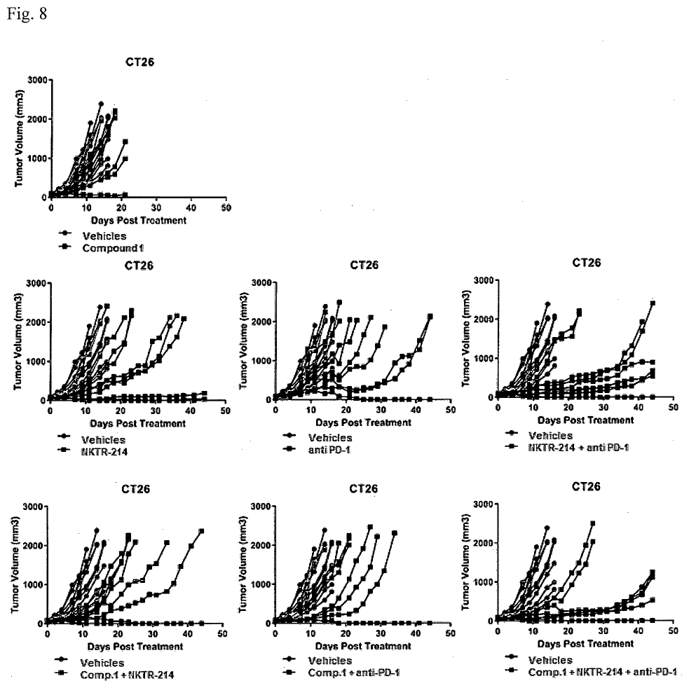

TABLE 9

| Results etc. of the syngeneic model study | | | | |
|---|---|---|---|---|
| | Inoculated | | | |
| Con- | tumor cell | Administration condition | | |
| comitant | (starting | | Concomitant | |
| drug | tumor size) | Compound 1 | drug | Result |
| Irinotecan | PHTXM- | 20 mg/kg | At 5 mg/kg, | Additive |
| (CPT-11) | 97Pa | once | intraperitoneally | |
| | (180 mm³) | daily on | on Days | |
| | | Day 1-21 | 1, 5, 9, 13, 17, 21 | |
| | | 40 mg/kg | At 5 mg/kg, | subadditive |
| | | once | intraperitoneally | |
| | | daily on | on Days | |
| | | Day 1-21 | 1, 5, 9, 13, 17, 21 | |
| | | 20 mg/kg | At 10 mg/kg, | Additive |
| | | once | intraperitoneally | |
| | | daily on | on Days | |
| | | Day 1-21 | 1, 5, 9, 13, 17, 21 | |
| | | 40 mg/kg | At 10 mg/kg, | subadditive |
| | | once | intraperitoneally | |
| | | daily on | on Days | |
| | | Day 1-21 | 1, 5, 9, 13, 17, 21 | |
| | PHTX- | 20 mg/kg | At 5 mg/kg, | Additive |
| | 249Pa | once | intraperitoneally | |
| | (195 mm³) | daily on | on Days | |
| | | Day 1-21 | 1, 5, 9, 13, 17, 21 | |
| | | 40 mg/kg | At 5 mg/kg, | Additive |
| | | once | intraperitoneally | |
| | | daily on | on Days | |
| | | Day 1-21 | 1, 5, 9, 13, 17, 21 | |
| | | 20 mg/kg | At 10 mg/kg, | Additive |
| | | once | intraperitoneally | |
| | | daily on | on Days | |
| | | Day 1-21 | 1, 5, 9, 13, 17, 21 | |
| | | 40 mg/kg | At 10 mg/kg, | Additive |
| | | once | intraperitoneally | |
| | | daily on | on Days | |
| | | Day 1-21 | 1, 5, 9, 13, 17, 21 | |
| | PHTXM- | 60 mg/kg | At 10 mg/kg, | Additive |
| | 35Es | once | intraperitoneally | |
| | (240 mm³) | daily on | on Days | |
| | | Day 1-21 | 1, 5, 9, 13, 17, 21 | |
| | PHTXM- | 60 mg/kg | At 10 mg/kg, | Additive |
| | 79Es | once | intraperitoneally | |
| | (160 mm³) | daily on | on Days | |
| | FIG. 5B | Day 1-21 | 1, 5, 9, 13, 17, 21 | |
| | PHTXM- | 60 mg/kg | At 10 mg/kg, | Synergistic |
| | 90Es | once | intraperitoneally | |
| | (185 mm³) | daily on | on Days | |
| | | Day 1-21 | 1, 5, 9, 13, 17, 21 | |
| Docetaxel | PHTXM- | 40 mg/kg | At 10 mg/kg,, | Synergistic |
| | 35Es | once | intravenously | |
| | (190 mm³) | daily on | once weekly on | |
| | | Day 1-21 | Days 1, 8, 15 | |
| | | 60 mg/kg | At 10 mg/kg,, | Synergistic |
| | | once | intravenously | |
| | | daily on | once weekly on | |
| | | Day 1-21 | Days 1, 8, 15 | |
| | PHTXM- | 40 mg/kg | At 10 mg/kg,, | Synergistic |
| | 35Es | once | intravenously | |
| | (150 mm³) | daily on | once weekly on | |
| | FIG. 4B | Day 1-21 | Days 1, 8, 15 | |
| | | 60 mg/kg | At 10 mg/kg,, | Additive |
| | | once | intravenously | |
| | | daily on | once weekly on | |
| | | Day 1-21 | Days 1, 8, 15 | |
| | PHTX- | 40 mg/kg | At 10 mg/kg, | Synergistic |
| | 79Es | once | intravenously | |
| | (135 mm³) | daily on | once weekly on | |
| | | Day 1-21 | Days 1, 8, 15 | |
| | FIG. 5A | 60 mg/kg | At 10 mg/kg, | additive |
| | | once | intravenously | |
| | | daily on | once weekly on | |
| | | Day 1-21 | Days 1, 8, 15 | |
| | PHTXM- | 40 mg/kg | At 10 mg/kg, | Additive |
| | 90Es | once | intravenously | |
| | (220 mm³) | daily on | once weekly on | |
| | | Day 1-21 | Days 1, 8, 15 | |
| | | 60 mg/kg | At 10 mg/kg, | Additive |
| | | once | intravenously | |
| | | daily on | once weekly on | |
| | | Day 1-21 | Days 1, 8, 15 | |
| | DU-145 | 60 mg/kg | At 10 mg/kg, | Additive |
| | (165 mm³) | once | intravenously | |
| | | daily on | once weekly on | |
| | | Day 1-21 | Days 1, 8, 15 | |
| Fluroracil | PHTX-11C | 40 mg/kg | At 15 mg/kg, | Additive |
| (5-FU) | (270 mm³) | once | intravenously | |
| | | daily on | on Days 1-3, | |
| | | Day 1-21 | 8-10, 15-17 | |
| | | 40 mg/kg | At 25 mg/kg, | Additive |
| | | once | intravenously | |
| | | daily on | on Days 1-3, | |
| | | Day 1-21 | 8-10, 15-17 | |
| | | 60 mg/kg | At 15 mg/kg, | Additive |
| | | once | intravenously | |
| | | daily on | on Days 1-3, | |
| | | Day 1-21 | 8-10, 15-17 | |
| | | 60 mg/kg | At 25 mg/kg, | subadditive |
| | | once | intravenously | |
| | | daily on | on Days 1-3, | |
| | | Day 1-21 | 8-10, 15-17 | |
| | PHTX-09C | 40 mg/kg | At 15 mg/kg, | Additive |
| | (155 mm³) | once | intravenously | |
| | | daily on | on Days 1-3, | |
| | | Day 1-21 | 8-10, 15-17 | |
| | | 40 mg/kg | At 25 mg/kg, | Additive |
| | | once | intravenously | |
| | | daily on | on Days 1-3, | |
| | | Day 1-21 | 8-10, 15-17 | |
| | | 60 mg/kg | At 15 mg/kg, | Additive |
| | | once | intravenously | |
| | | daily on | on Days 1-3, | |
| | | Day 1-21 | 8-10, 15-17 | |
| | | 60 mg/kg | At 25 mg/kg, | subadditive |
| | | once | intravenously | |
| | | daily on | on Days 1-3, | |
| | | Day 1-21 | 8-10, 15-17 | |
| | PHTXM- | 60 mg/kg | At 25 mg/kg, | Additive |
| | 35Es | once | intravenously | |
| | (240 mm³) | daily on | on Days 1-3, | |
| | | Day 1-21 | 8-10, 15-17 | |
| | PHTXM- | 60 mg/kg | At 25 mg/kg, | Synergistic |
| | 79Es | once | intravenously | |
| | (160 mm³) | daily on | on Days 1-3, | |
| | | Day 1-21 | 8-10, 15-17 | |
| | PHTXM- | 60 mg/kg | At 25 mg/kg, | Additive |
| | 90Es | once | intravenously | |
| | (185 mm³) | daily on | on Days 1-3, | |
| | | Day 1-21 | 8-10, 15-17 | |
| Gem- | DU-145 | 60 mg/kg | At 40 mg/kg, | Additive |
| citabine | (165 mm³) | once | intraperitoneally | |
| | | daily on | on Days | |
| | | Day 1-21 | 1, 4, 8, | |
| | | | 11, 15, 18 | |
| | PHTX- | 40 mg/kg | At 20 mg/kg, | Additive |
| | 249Pa | once | intraperitoneally | |
| | (165 mm³) | daily on | on Days | |
| | | Day 1-21 | 1, 4, 8, | |
| | | | 11, 15, 18 | |
| | | 60 mg/kg | At 20 mg/kg, | Additive |
| | | once | intraperitoneally | |
| | | daily on | on Days | |
| | | Day 1-21 | 1, 4, 8, | |
| | | | 11, 15, 18 | |
| | | 40 mg/kg | At 40 mg/kg, | subadditive |
| | | once | intraperitoneally | |
| | | daily on | on Days | |
| | | Day 1-21 | 1, 4, 8, | |
| | | | 11, 15, 18 | |
| | | 60 mg/kg | At 40 mg/kg, | subadditive |
| | | once | intraperitoneally | |
| | | daily on | on Days | |
| | | Day 1-21 | 1, 4, 8, | |
| | | | 11, 15, 18 | |
| | PHTX- | 20 mg/kg | At 5 mg/kg, | Synergistic |
| | 249Pa | once | intraperitoneally | |
| | (180 mm³) | daily on | on Days | |
| | FIG. 6A | Day 1-21 | 1, 4, 8, | |
| | | | 11, 15, 18 | |
| | | 40 mg/kg | At 5 mg/kg, | Additive |
| | | once | intraperitoneally | |
| | | daily on | on Days | |
| | | Day 1-21 | 1, 4, 8, | |
| | | | 11, 15, 18 | |
| | | 20 mg/kg | At 20 mg/kg, | Additive |
| | | once | intraperitoneally | |
| | | daily on | on Days | |
| | | Day 1-21 | 1, 4, 8, | |
| | | | 11, 15, 18 | |
| | | 40 mg/kg | At 20 mg/kg, | subadditive |
| | | once | intraperitoneally | |
| | | daily on | on Days | |
| | | Day 1-21 | 1, 4, 8, | |
| | | | 11, 15, 18 | |
| | PHTXM- | 40 mg/kg | At 20 mg/kg, | subadditive |
| | 97Pa | once | intraperitoneally | |
| | (210 mm³) | daily on | on Days | |
| | | Day 1-21 | 1, 4, 8, | |
| | | | 11, 15, 18 | |
| | | 60 mg/kg | At 20 mg/kg, | subadditive |
| | | once | intraperitoneally | |
| | | daily on | on Days | |
| | | Day 1-21 | 1, 4, 8, | |
| | | | 11, 15, 18 | |
| | | 40 mg/kg | At 40 mg/kg, | subadditive |
| | | once | intraperitoneally | |
| | | daily on | on Days | |
| | | Day 1-21 | 1, 4, 8, | |
| | | | 11, 15, 18 | |
| | | 60 mg/kg | At 40 mg/kg, | subadditive |
| | | once | intraperitoneally | |
| | | daily on | on Days | |
| | | Day 1-21 | 1, 4, 8, | |
| | | | 11, 15, 18 | |
| | PHTXM- | 20 mg/kg | At 5 mg/kg, | subadditive |
| | 97Pa | once | intraperitoneally | |
| | (180 mm³) | daily on | on Days | |
| | | Day 1-21 | 1, 4, 8, | |
| | | | 11, 15, 18 | |
| | | 40 mg/kg | At 5 mg/kg, | subadditive |
| | | once | intraperitoneally | |
| | | daily on | on Days | |
| | | Day 1-21 | 1, 4, 8, | |
| | | | 11, 15, 18 | |
| | | 20 mg/kg | At 20 mg/kg, | Additive |
| | | once | intraperitoneally | |
| | | daily on | on Days | |
| | | Day 1-21 | 1, 4, 8, | |
| | | | 11, 15, 18 | |
| | | 40 mg/kg | At 20 mg/kg, | subadditive |
| | | once | intraperitoneally | |
| | | daily on | on Days | |
| | | Day 1-21 | 1, 4, 8, | |
| | | | 11, 15, 18 | |
| | Panc02 | 60 mg/kg | At 5 mg/kg, | Additive |
| | (75 mm³) | orally, | intraperitoneally, | |
| | | QD on | QD on Day | |
| | | day 0-20 | 0, 3, 6, | |
| | | | 9, 12, | |
| Carboplatin | PHTXS- | 40 mg/kg | At 30 mg/kg, | Additive |
| | 13O | once | intraperitoneally | |
| | (170 mm³) | daily on | on Days | |
| | FIG. 4A | Day 1-14 | 1, 5, 9, 13 | |
| | | 60 mg/kg | At 30 mg/kg, | Additive |
| | | once | intraperitoneally | |
| | | daily on | on Days | |
| | | Day 1-14 | 1, 5, 9, 13 | |
| | | 40 mg/kg | At 50 mg/kg, | Additive |
| | | once | intraperitoneally | |
| | | daily on | on Days | |
| | | Day 1-14 | 1, 5, 9, 13 | |
| | | 60 mg/kg | At 50 mg/kg, | Additive |
| | | once | intraperitoneally | |
| | | daily on | on Days | |
| | | Day 1-14 | 1, 5, 9, 13 | |
| Palbociclib | PHTXS- | 40 mg/kg | At 50 mg/kg, | Additive |
| | 13O | once | orally on | |
| | (110 mm³) | daily on | Days 1-21 | |
| | FIG. 6B | Day 1-21 | | Additive |
| | | 60 mg/kg | At 50 mg/kg, | |
| | | once | orally on | |
| | | daily on | Days 1-21 | |
| | | Day 1-21 | | Additive |
| | | 40 mg/kg | At 100 mg/kg, | |
| | | once | orally on | |
| | | daily on | Days 1-21 | |
| | | Day 1-21 | | |
| | | 60 mg/kg | At 100 mg/kg, | Additive |
| | | once | orally on | |
| | | daily on | Days 1-21 | |
| | | Day 1-21 | | Additive |
| | PHTXS- | 60 mg/kg | At 100 mg/kg, | |
| | 13O | once | orally on | |
| | (120 mm³) | daily on | Days 1-14 | |
| | | Day 1-14 | | |
| Anti- | CT26 | 60 mg/kg | At 200 | Additive |
| mPD-1 | (64 mm³) | orally, | ug/mouse, | |
| | | QD × 3/wk | intraperitoneally | |
| | | on day | on Day | |
| | | 0, 1, 2, | 0, 3, 7, | |
| | | 7, 8, 9, | 10, 14, 17 | |
| | | 14, 15, 16 | | |
| | B16F10 | 60 mg/kg | At 200 | Additive |
| | (67 mm³) | orally, | ug/mouse, | |
| | | QD × 3/wk | intraperitoneally | |
| | | on day | on Day | |
| | | 0, 1, 2, | 0, 3, 7, | |
| | | 7, 8, 9 | 10 | |
| | A20 | 60 mg/kg | At 200 | Additive |
| | (61 mm³) | orally, | ug/mouse, | |
| | | QD × 3/wk | intraperitoneally | |
| | | on day | on Day | |
| | | 0, 1, 2, | 0, 3, 7, | |
| | | 7, 8, 9, | 10, 14, 17 | |
| | | 14, 15, 16 | | |
| | Panc02 | 60 mg/kg | At 200 | Additive |
| | (75 mm³) | orally, | ug/mouse, | |
| | | QD on | intraperitoneally | |
| | | day 1-21 | on Day | |
| | | | 0, 3, 7, 10 | |
| | CT26 | 60 mg/kg | At 10 | Additive |
| | (50 mm³) | orally, | mg/kg, | |
| | | QD on | intraperitoneally | |
| | | day 0-20 | on Day | |
| | | | 0, 3, 7, | |
| | | | 10, 14, 17 | |
| | CT26 | 60 mg/kg | At 10 | Additive |
| | (40 mm³) | orally, | mg/kg, | |
| | FIG. 8 | QD on | intraperitoneally | |
| | | day 0-20 | on Day | |
| | | | 0, 3, 7, | |
| | | | 10, 14, 17 | |
| | JC | 60 mg/kg | At 10 | Additive |
| | (60 mm³) | orally, | mg/kg, | |
| | | QD on | intraperitoneally | |
| | | day 0-20 | on Day | |
| | | | 0, 3, 7, | |
| | | | 10, 14, 17 | |
| | J558 | 60 mg/kg | At 10 | Additive |
| | (30 mm³) | orally, | mg/kg, | |
| | FIG. 7 | QD on | intraperitoneally | |
| | | day 0-20 | on Day | |
| | | | 0, 3, 7, | |
| | | | 10, 14, 17 | |
| NKTR-214 | CT26 | 60 mg/kg | At 0.8 mg/kg, | Additive |
| | (64 mm³) | orally, | intravenously, | |
| | FIG. 8 | QD × 3/ | Q9D on Day | |
| | | wk on day | 0, 9, 18 | |
| | | 0, 1, 2, | | |
| | | 7, 8, 9, | | |
| | | 14, 15, 16 | | |
| | B16F10 | 60 mg/kg | At 0.8 mg/kg, | Additive |
| | (67 mm³) | orally, | intravenously, | |
| | | QD × 3/ | Q9D on Day | |
| | | wk on day | 0, 9, 18 | |
| | | 0, 1, 2, | | |
| | | 7, 8, 9 | | |
| | A20 | 60 mg/kg | At 0.8 mg/kg, | Additive |
| | (61 mm³) | orally, | intravenously, | |
| | | QD × 3/ | Q9D on Day | |
| | | wk on day | 0, 9, 18 | |
| | | 0, 1, 2, | | |
| | | 7, 8, 9, | | |
| | | 14, 15, 16 | | |
| NKTR- | CT26 | 60 mg/kg | NKTR-214 | Additive |
| 214 + | (64 mm³) | orally, | At 0.8 mg/kg, | |
| anti- | FIG. 8 | QD × 3/ | intravenously, | |
| mPD-1 | | wk on day | Q9D on Day | |
| | | 0, 1, 2, | 0, 9, 18; and | |
| | | 7, 8, 9, | anti-mPD-1 | |
| | | 14, 15, 16 | Ab. at 200 | |
| | | | ug/mouse, | |
| | | | intraperitoneally, | |
| | | | on Day | |
| | | | 0, 3, 7, | |
| | | | 10, 14, 17 | |
| | B16F10 | 60 mg/kg | NKTR-214 | Additive |
| | (67 mm³) | orally, | At 0.8 mg/kg, | |
| | | QD × 3/ | intravenously, | |
| | | wk on day | Q9D on Day | |
| | | 0, 1, 2, | 0, 9, 18; and | |
| | | 7, 8, 9 | anti-mPD-1 | |
| | | | Ab. at 200 | |
| | | | ug/mouse, | |
| | | | intraperitoneally, | |
| | | | on Day | |
| | | | 0, 3, 7, | |
| | | | 10, 14, 17 | |
| | A20 | 60 mg/kg | NKTR-214 | Additive |
| | (61 mm³) | orally, | At 0.8 mg/kg, | |
| | | QD × 3/ | intravenously, | |
| | | wk on day | Q9D on Day | |
| | | 0, 1, 2, | 0, 9, 18; and | |
| | | 7, 8, 9, | anti-mPD-1 | |
| | | 14, 15, 16 | Ab. at 200 | |
| | | | ug/mouse, | |
| | | | intraperitoneally, | |
| | | | on Day | |
| | | | 0, 3, 7, | |
| | | | 10, 14, 17 | |
| Anti- | CT26 | 60 mg/kg | At 10 mg/kg, | Additive |
| mCTLA-4 | (50 mm³) | orally, | intraperitoneally | |
| | | QD on | on Day | |
| | | day 0-20 | 0, 3, 7, | |
| | | | 10, 14, 17 | |
| | CT26 | 60 mg/kg | At 10 mg/kg, | Additive |
| | (40 mm³) | orally, | intraperitoneally | |
| | | QD on | on Day | |
| | | day 0-20 | 0, 3, 7, | |
| | | | 10, 14, 17 | |
| | JC | 60 mg/kg | At 10 mg/kg, | Additive |
| | (60 mm³) | orally, | intraperitoneally | |
| | | QD on | on Day | |
| | | day 0-20 | 0, 3, 7, | |
| | | | 10, 14, 17 | |
| | J558 | 60 mg/kg | At 10 mg/kg, | Additive |
| | (30 mm³) | orally, | intraperitoneally | |
| | FIG. 7 | QD on | on Day | |
| | | day 0-20 | 0, 3, 7, | |
| | | | 10, 14, 17 | |
| | H22 | 60 mg/kg | At 3 mg/kg, | Additive |
| | (55 mm³) | orally, | intraperitoneally | |
| | | QD on | on Day | |
| | | day 0-20 | 0, 3, 7, | |
| | | | 10, 14, 17 | |
| Anti- | J558 | 60 mg/kg | At 10 mg/kg, | Additive |
| mPD-L1 | (30 mm³) | orally, | intraperitoneally | |
| | FIG. 7 | QD on | on Day | |
| | | day 0-20 | 0, 3, 7, | |
| | | | 10, 14, 17 | |

Note:
0 CR in Gemcitabine group and 0 CR in Compound 1 group, and 0 CR in Compound 1/Gemcitabine combo group
Note:
Several mice in the group suffered Body Weight Loss during drug treatment
Note:
1 CR in PD-1 group and no CR in Compound 1 group, and 2 CR in Compound 1/PD-1 combo group
Note:
0 CR in PD-1 group and 0 CR in Compound 1 group, and 0 CR in Compound 1/PD-1 combo group
Note:
4 CR in PD-1 group and 1 CR in Compound 1 group, and 3 CR in Compound 1/PD-1 combo group
Note:
0 CR in PD-1 group and 0 CR in Compound 1 group, and 0 CR in Compound 1/PD-1 combo group
Note:
0 CR in PD-1 group and 0 CR in Compound 1 group, and 1 CR in Compound 1/PD-1 combo group
Note:
2 CR in PD-1 group and 0 CR in Compound 1 group, and 2 CR in Compound 1/PD-1 combo group
Note:
0 CR in PD-1 group and 0 CR in Compound 1 group, and 0 CR in Compound 1/PD-1 combo group
Note:
2 CR in PD-1 group and 2 CR in Compound 1 group, and 7 CR in Compound 1/PD-1 combo group
Note:
0 CR in NKTR-214 group and no CR in Compound 1 group, and 2 CR in Compound 1/NKTR-214 combo group
Note:
0 CR in NKTR-214 group and no CR in Compound 1 group, and 0 CR in Compound 1/NKTR-214 combo group
Note:
3 CR in NKTR-214 group and 1 CR in Compound 1 group, and 2 CR in Compound 1/NKTR-214 combo group
Note:
2 CR in NKTR-214 + PD-1 group, and no CR in Compound 1 group, and 4 CR in Compound 1/NKTR-214/PD-1 triple combo group
Note:
0 CR in NKTR-214 + PD-1 group, and 0 CR in Compound 1 group, and 0 CR in Compound 1/NKTR-214/PD-1 triple combo group
Note:
8 CR in NKTR-214 + PD-1 group, and 1 CR in Compound 1 group, and 5 CR in Compound 1/NKTR-214/PD-1 triple combo group
Note:
5 CR in CTLA-4 group and 0 CR in Compound 1 group, and 5 CR in Compound 1/CTLA-4 combo group
Note:
8 CR in CTLA-4 group and 0 CR in Compound 1 group, and 8 CR in Compound 1/CTLA-4 combo group
Note:
0 CR in CTLA-4 group and 0 CR in Compound 1 group, and 0 CR in Compound 1/CTLA-4 combo group
Note:
1 CR in CTLA-4 group and 2 CR in Compound 1 group, and 7 CR in Compound 1/CTAL-4 combo group
Note:
9 CR in CTLA-4 group and 0 CR in Compound 1 group, and 8 CR in Compound 1/CTAL-4 combo group
Note:
1 CR in PD-L1 group and 2 CR in Compound 1 group, and 3 CR in Compound 1/PD-L1 combo group

### Example 6

To discover potential genes sensitizing with compound 1 treatment, CRISPR-Cas9 knock-out screening was performed at Horizon Discovery Ltd (Cambridge, UK). Twelve cancer cell lines (A549, BxPC3, Calu-1, COLO205, KYSE140, KYSE150, KYSE520, KYSE70, MIA PaCa-2, NCI-H292, PANC1, and RKO) and custom gRNA library for 1969 genes were used for the screening.

Cells were treated with lentivirus containing gRNAs and Cas-9 for 2 hours, and then the cells were re-suspended in flesh medium. After 48 hours recovery period, Puromycin was added to select cells. Following completion of selection, the cells were maintained in culture medium containing DMSO, low dose compound 1, or high dose compound 1. The dose of compound 1 was adjusted at each passage to maintain appropriate selective pressure. After 12 population doubling of the DMSO-treated cells, cells were harvested and stored in a deep freezer. Genomic DNA of the cells were extracted. Samples were prepared and purified for amplicon sequencing using an Illumina NextSeq next generation sequencing (NGS) platform. Analysis of NGS data sets was achieved using Horizon's data processing scripts. The data was analyzed using following formula to calculate enrichment score of each gene and its p-value.

Enrichment Score (ES)=log 2(compound 1+guide i)+log 2(control+dummyguide)−log 2(compound 1+dummyguide)−log 2(control+guide i)

Genes with ES<0 and p-value <0.05 in comparison between control (DMSO treated) and low dose compound 1 treated cells were defined as a sensitizing hit genes. Following genes were identified as sensitizing hit gene in more than three cancer cell lines; ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1, ZNF638.

This experiment revealed that mutation or deletion of at least one gene of above hit genes rendered cancer cells more sensitive to compound 1.

### Example 7

To determine further whether RNASEH2A is involved in the sensitization to compound 1, in vitro growth inhibition assay of compound 1 was carried out in RNASEH2A knockout (KO) TK-6 cells and its counterpart parental TK-6 cells. RNASEH2A KO TK-6 cells and its counterpart parental TK-6 cells were obtained from Kyoto University under a material transfer agreement. The cell lines were cultured in RPMI-1640 medium (FUJIFILM Wako Pure Chemical Corporation, Osaka, JAPAN) supplied with 10% Fetal Bovine Serum (CORNING Inc., NY, USA), sodium pyruvate (FUJIFILM Wako Pure Chemical Corporation, Osaka, JAPAN), and Penicillin-Streptomycin (FUJIFILM Wako Pure Chemical Corporation, Osaka, JAPAN). Stock solutions of compound 1 were prepared in dimethyl sulfoxide (DMSO), and stored at approximately −20 deg C.

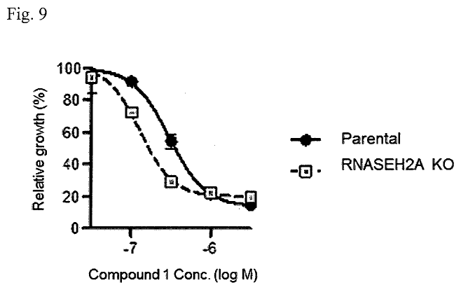

Cell proliferation was measured by using Cell Titer-Glo Luminescent Cell Viability Assay (Promega, WI, USA.). The CellTiter-Glo Luminescent Cell Viability Assay is a homogeneous method of determining the number of viable cells in culture based on quantitation of the ATP present, which signals the presence of metabolically active cells. Compound 1 was diluted and the solutions were plated in a 384 well plate at 20 μL/well. Then, 20 μL of the cells in the culture medium were sown to adjust a final density at 500 cells/well, and cultured in an incubator (37° C., 5% carbon dioxide). After incubation for 72 hours, 20 μL of solution of Cell Titer-Glo Luminescent Cell Viability Assay was added to each well and incubated for approximately 30 min at room temperature. Luminescence of each well was measured by EnVision™ (PerkinElmer Inc., MA, USA). Taking as 100% the ATP content for the DMSO treatment control group, the ratio of the residual ATP content for each treatment group was determined. Growth inhibition curve of Compound 1 in RNASEH2A KO TK-6 cells and its counter partner parental TK-6 cells were described using GraphPad Prism (GraphPad Software, Inc., CA, USA.), and is shown in FIG. 9. This experiment revealed that RNASEH2A KO TK-6 cells were more sensitive to compound 1 than WT TK-6 cells.

1. A method for treating cancer in a patient in need thereof, comprising administering to the patient a therapeutically effective amount of Compound 1 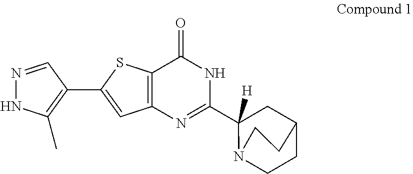 [SMILES: Cc1[nH]ncc1-c1cc2nc([C@@H]3CC4CCN3CC4)[nH]c(=O)c2s1]
and/or tautomers thereof or a pharmaceutically acceptable salt or hydrate thereof; and one or more DNA damaging agents selected from mitomycin C, teniposide, topotecan hydrochloride, carboplatin, decitabine, and melphalan.

2. The method of claim 1, wherein the cancer can repair homologous recombination in the cancer cell.

3. The method of claim 1, wherein the cancer is platinum compound-resistant.

4. The method of claim 1, wherein the cancer is lung cancer, colorectal cancer, pancreatic cancer, or ovarian cancer.

5. A method for treating cancer in a patient in need thereof, comprising administering to the patient a therapeutically effective amount of Compound 1 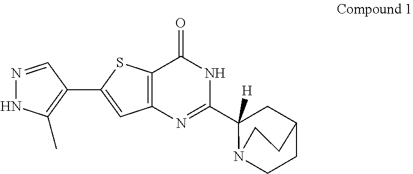 [SMILES: Cc1[nH]ncc1-c1cc2nc([C@@H]3CC4CCN3CC4)[nH]c(=O)c2s1]
and/or tautomers thereof or a pharmaceutically acceptable salt or hydrate thereof, wherein the patient has mutation or deletion in one or more gene selected from a group consisting of RNASEH2A, RNASEH2B and RNASEH2C.

6. The method of claim 5, wherein the cancer is lung cancer, colorectal cancer, pancreatic cancer, or ovarian cancer.

7. A method for treating cancer in a patient comprising (1) determining whether the patient has the mutation and/or deletion status by (a) obtaining or having obtained a biological sample from the patient; (b) performing or having performed an assay on the biological samples to reveal if the patient has one or more mutated and/or deleted genes; (2) if the patient has the mutation and/or deletion status, then administering to the patient a therapeutically effective amount of Compound 1 and/or tautomers thereof or a pharmaceutically acceptable salt or hydrate thereof; wherein the mutation and/or deletion gene is selected from ALKBH6, APEX1, APEX2, ARFGEF1, ASF1A, ASF1B, ATRX, BAZ1B, C21orf2, CAV1, CDC25B, CDK19, CDKN1B, CNOT2, CNOT4, DBF4, DDX5, E2F4, ERCC4, ESCO2, FAF1, FANCD2, FANCG, FANCI, FANCL, FBXO5, FBXW7, FOXM1, GMNN, HIST1H3G, IKZF2, ITGB6, KMT2E, KPNA2, MAD2L2, MAP3K7, MLLT1, MTBP, NAE1, NHEJ1, POLA2, POT1, PPP2R5D, PPP4R2, PSMC3IP, PUS1, RAD54L, RFWD3, RNASEH2A, RNASEH2B, RNASEH2C, RNF8, RTEL1, SMARCA4, STK11, TAOK3, TICRR, TIPIN, UBE2A, UBE2C, UHRF1, UNG, USP1, USP37, USP7, VRK1, WEE1, XRCC1 and ZNF638.

8. The method of claim 7, wherein the mutation and/or deletion gene is selected from RNASEH2A, RNASEH2B and RNASEH2C.

9. The method of claim 7, further comprising one or more DNA damaging agents selected from mitomycin C, teniposide, topotecan hydrochloride, carboplatin, decitabine, and melphalan.

10. The method of claim 7, wherein the cancer is lung cancer, colorectal cancer, pancreatic cancer, or ovarian cancer.

11. A use of Compound 1 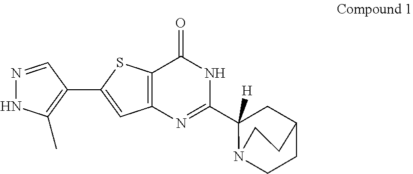 [SMILES: Cc1[nH]ncc1-c1cc2nc([C@@H]3CC4CCN3CC4)[nH]c(=O)c2s1]
and/or tautomers thereof or a pharmaceutically acceptable salt or hydrate thereof for the manufacture of a medicament for use in combination with one or more DNA damaging agents for the treatment of cancer, wherein the DNA damaging agent comprises mitomycin C, teniposide, topotecan hydrochloride, carboplatin, decitabine, or melphalan.

12. Compound 1 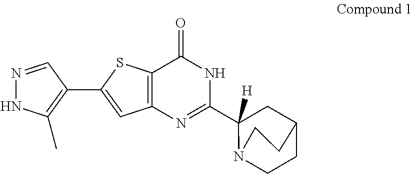 [SMILES: Cc1[nH]ncc1-c1cc2nc([C@@H]3CC4CCN3CC4)[nH]c(=O)c2s1]
and/or tautomers thereof or a pharmaceutically acceptable salt or hydrate thereof for use in combination with one or more DNA damaging agents in the treatment of cancer, wherein the DNA damaging agent is selected from a group consisting of mitomycin C, teniposide, topotecan hydrochloride, carboplatin, decitabine, and melphalan.
